(19) United States
(12) Patent Application Publication
(10) Pub. No.: US 2012/0171693 A1
(43) Pub. Date: Jul. 5, 2012

(54) Methods for Generating Novel Stabilized Proteins

(75) Inventors: Frances H. Arnold, La Canada, CA (US); Yougen Li, Lawrenceville, NJ (US)

(73) Assignee: THE CALIFORNIA INSTITUTE OF TECHNOLOGY, Pasadena, CA (US)

(21) Appl. No.: 11/969,894

(22) Filed: Jan. 5, 2008

Related U.S. Application Data
(60) Provisional application No. 60/878,962, filed on Jan. 5, 2007.
(60) Provisional application No. 60/899,120, filed on Feb. 2, 2007.
(60) Provisional application No. 60/900,229, filed on Feb. 8, 2007.
(60) Provisional application No. 60/918,528, filed on Mar. 16, 2007.

(51) Int. Cl. C12Q 1/68; C12Q 1/34; C12Q 1/40; G06F 19/20; C12Q 1/26; C12Q 1/28; C12N 9/00; G06F 19/14; C12Q 1/48; C12Q 1/37

(52) U.S. Cl. 435/6.18; 435/15; 435/18; 435/22; 435/23; 435/24; 435/25; 435/28; 435/183; 702/19; 702/20

(57) ABSTRACT

The disclosure provides methods for identifying and producing stabilized chimeric proteins.

### CROSS-REFERENCE TO RELATED APPLICATIONS

This application claims the benefit of U.S. Provisional Application Ser. Nos. 60/878,962, filed Jan. 5, 2007; 60/899,120, filed Feb. 2, 2007; 60/900,229, filed Feb. 8, 2007; and 60/918,528, filed, Mar. 16, 2007 the disclosures of which are incorporated herein by reference.

### STATEMENT REGARDING FEDERALLY SPONSORED RESEARCH

The U.S. Government has certain rights in this invention pursuant to Grant No. GM068664 awarded by the National Institutes of Health and Grant No. DAAD19-03-0D-0004 awarded by ARO-US Army Robert Morris Acquisition Center.

### FIELD OF THE INVENTION

The invention relates to biomolecular engineering and design, including methods for the design and engineering of biopolymers such as proteins and nucleic acids.

### BACKGROUND

A repertoire of stable proteins that can be further refined for research, industry and medical use is important.

### SUMMARY

The disclosure provides a method for generating one or more stabilized proteins. The disclosure uses regression analysis to determine those segments that contribute to protein stability. Recombinant chimeric proteins that demonstrate stability are analyzed to determine their chimeric components. The regression analysis comprises determining sequence-stability data and the consensus analysis comprises determining multiple sequence alignment (MSA) of folded versus unfolded proteins.

The disclosure includes a method comprising identifying a set of structurally or evolutionarily related polypeptides and their corresponding polynucleotide sequences; aligning their sequences based on structure similarity; selecting a set of 2 or more crossover locations in the aligned sequences; recombinantly producing and testing a set of representative proteins (e.g., a set of xP^{N} possible recombined sequences, wherein P is the number of parent proteins, N is the number of segments and x<1); expressing the proteins encoded by those sequences; measuring the stabilities of those sequences; analyzing the relationship between sequence and stability; predicting the most stable sequences from the set using regression analysis and/or consensus analysis; and testing those proteins to confirm stability and bioactivity.

The disclosure provides a method for generating one or more stabilized proteins, comprising: identifying a plurality (P) of evolutionary, structurally or evolutionary and structurally related polypeptides; selecting a set of crossover locations comprising N peptide segments in at least a first polypeptide and at least a second polypeptide of the plurality of related polypeptides; generating a sample set (xP^{N}) of recombined, recombinant proteins comprising peptide segments from each of the at least first polypeptide and second polypeptide, wherein x<1; measuring stability of the sample set of expressed-folded recombined, recombinant proteins; performing regression analysis and/or consensus analysis of recombined, recombinant proteins having stability to identify stability-associated peptide segments; generating a stabilized polypeptide comprising the stability-associated peptide segment; and measuring the activity and/or stability of the stabilized polypeptide. The stabilized protein can comprise any number of enzymes or proteins including, for example, P450's, carbohydrases, alpha-amylase, β-amylase, cellulase, β-glucanase, β-glucosidase, dextranase, dextrinase, glucoamylase, hemmicellulase/pentosanase/xylanase, invertase, lactase, pectinase, pullulanase, proteases, oxygenases, acid proteinase, alkaline protease, pepsin, peptidases, aminopeptidase, endo-peptidase, subtilisin, lipases and esterases, aminoacylase, glutaminase, lysozyme, penicillin acylase, isomerase, oxireductases, alcohol dehydrogenase, amino acid oxidase, catalase, chloroperoxidase, peroxidase, lyases, acetolactate decarboxylase, aspartic β-decarboxylase, histidase, transferases, and cyclodextrin glycosyltransferase. In one aspect, the selecting a set of crossover locations comprises: aligning the sequences of the plurality of evolutionary, structurally or evolutionary and structurally related polypeptides; and identifying regions of identity of the sequences. In a further aspect, the method comprises sequence alignment and one or more methods selected from the group consisting of X-ray crystallography, NMR, searching a protein structure database, homology modeling, de novo protein folding, and computational protein structure prediction. In another aspect, the selecting a set of crossover locations comprises: identifying coupling interactions between pairs of residues in the at least first polypeptide; generating a plurality of data structures, each data structure representing a crossover mutant comprising a recombination of the at least first and second polypeptide, wherein each recombination has a different crossover location; determining, for each data structure, a crossover disruption related to the number of coupling interactions disrupted in the crossover mutant represented by the data structure; and identifying, among the plurality of data structures, a particular data structure having a crossover disruption below a threshold, wherein the crossover location of the crossover mutant represented by the particular data structure is the identified crossover location. In a further aspect, the coupling interactions are identified by a determination of a conformational energy between residues or by a determination of interatomic distances between residues. In another aspect, the conformation energies are determined from a three-dimensional structure for at least one of a first and second polypeptide. In another aspect, the interatomic distances are determined from a three-dimensional structure of at least one polypeptide of the plurality of polypeptides. In yet another aspect, the coupling interactions are identified by a conformational energy between residues above a threshold. In one aspect, the threshold is an average level of crossover disruption for the plurality of data structures. The identification of crossover location comprises identification of possible cut points in the polypeptide based upon regions of sequence identity. In one aspect, the measuring of stability comprises a techniques selected from the group consisting of chemical stability measurements, functional stability measurements and thermal stability measurements. The method includes regression analysis comprising determining sequence-stability data or consensus analysis comprising determining multiple sequence alignment (MSA) of folded versus unfolded proteins. In one aspect, the sequence-stability analysis can be expressed as:

$${T}_{50} = {a}_{0} + {\sum }_{i} {\sum }_{j} {a}_{ij} {x}_{ij} ,$$

where T₅₀ is the dependent variable and peptide segments xᵢⱼ (from the i^{th} position and j^{th} parent are the independent variables, wherein the constant term (a₀) is the predicted T₅₀ of a parental polypeptide and the regression coefficients aᵢⱼ represent the thermostability contributions of peptide segment xᵢⱼ relative to the corresponding reference peptide segment of the parental polypeptide. In another aspect, the consensus analysis comprises sequence information of stabilized polypeptides and a frequency of stability-associated peptide segments. The consensus analysis comprises measuring the frequency of a stability-associated peptide segment at a position (i) in a stabilized protein and exponentially valuing the position:segment repeats to give a consensus energy value. In one aspect, the stability-associated peptide segments that promote stability reduce the overall consensus energy value of a stabilized protein expressed as

$${Δɛ}_{total} ∝ {\sum }_{i} - ln \frac{{f}_{i}}{{f}_{i , ref}} .$$

In one aspect, the analysis comprises a combination of sequence-stability data and consensus analysis of multiple sequence alignment (MSA) of folded versus unfolded proteins.

The disclosure further provides a method for generating one or more stabilized proteins, comprising: selecting crossover locations in a set, P, of parental polynucleotides encoding polypeptides that are evolutionary, structurally or evolutionary and structurally related, wherein the set of crossover locations defines N oligonucleotide segments each segment encoding a peptide; performing recombination between a subset, xP^{N}, of the parental polynucleotides having crossover locations to obtain a sample set of recombined, recombinant proteins comprising peptide segments encoded by the oligonucleotide segments, wherein x<1; measuring stability of the sample set of expressed folded recombined, recombinant proteins; performing regression analysis and/or consensus analysis of recombined, recombinant proteins having stability to identify stability-associated peptide segments and the encoding oligonucleotide segment; generating a stabilized polypeptide encoded by a combination of oligonucleotide encoding stability-associated peptide segments; and measuring the activity and/or stability of the stabilized polypeptide. The stabilized protein can comprise any number of enzymes or proteins including, for example, P450's, carbohydrases, alpha-amylase, β-amylase, cellulase, β-glucanase, β-glucosidase, dextranase, dextrinase, glucoamylase, hemmicellulase/pentosanase/xylanase, invertase, lactase, pectinase, pullulanase, proteases, oxygenases, acid proteinase, alkaline protease, pepsin, peptidases, aminopeptidase, endo-peptidase, subtilisin, lipases and esterases, aminoacylase, glutaminase, lysozyme, penicillin acylase, isomerase, oxireductases, alcohol dehydrogenase, amino acid oxidase, catalase, chloroperoxidase, peroxidase, lyases, acetolactate decarboxylase, aspartic β-decarboxylase, histidase, transferases, and cyclodextrin glycosyltransferase. In one aspect, the selecting a set of crossover locations comprises: aligning the sequences of the plurality of evolutionary, structurally or evolutionary and structurally related polypeptides; and identifying regions of identity of the sequences. In a further aspect, the method comprises sequence alignment and one or more methods selected from the group consisting of X-ray crystallography, NMR, searching a protein structure database, homology modeling, de novo protein folding, and computational protein structure prediction. In another aspect, the selecting a set of crossover locations comprises: identifying coupling interactions between pairs of residues in the at least first polypeptide; generating a plurality of data structures, each data structure representing a crossover mutant comprising a recombination of the at least first and second polypeptide, wherein each recombination has a different crossover location; determining, for each data structure, a crossover disruption related to the number of coupling interactions disrupted in the crossover mutant represented by the data structure; and identifying, among the plurality of data structures, a particular data structure having a crossover disruption below a threshold, wherein the crossover location of the crossover mutant represented by the particular data structure is the identified crossover location. In a further aspect, the coupling interactions are identified by a determination of a conformational energy between residues or by a determination of interatomic distances between residues. In another aspect, the conformation energies are determined from a three-dimensional structure for at least one of a first and second polypeptide. In another aspect, the interatomic distances are determined from a three-dimensional structure of at least one polypeptide of the plurality of polypeptides. In yet another aspect, the coupling interactions are identified by a conformational energy between residues above a threshold. In one aspect, the threshold is an average level of crossover disruption for the plurality of data structures. The identification of crossover location comprises identification of possible cut points in the polypeptide based upon regions of sequence identity. In one aspect, the measuring of stability comprises a techniques selected from the group consisting of chemical stability measurements, functional stability measurements and thermal stability measurements. The method includes analysis comprising determining sequence-stability data or consensus analysis of multiple sequence alignment (MSA) of folded versus unfolded proteins. In one aspect, the sequence-stability analysis can be expressed as:

$${T}_{50} = {a}_{0} + {\sum }_{i} {\sum }_{j} {a}_{ij} {x}_{ij} ,$$

where T₅₀ is the dependent variable and peptide segments xᵢⱼ (from the i^{th} position and j^{th} parent are the independent variables, wherein the constant term (a₀) is the predicted T₅₀ of a parental polypeptide and the regression coefficients aᵢⱼ represent the thermostability contributions of peptide segment xᵢⱼ relative to the corresponding reference peptide segment of the parental polypeptide. In another aspect, the consensus analysis comprises sequence information of stabilized polypeptides and a frequency of stability-associated peptide segments. The consensus analysis comprises measuring the frequency of a stability-associated peptide segment at a position (i) in a stabilized protein and exponentially valuing the position:segment repeats to give a consensus energy value. In one aspect, the stability-associated peptide segments that promote stability reduce the overall consensus energy value of a stabilized protein expressed as

$${Δɛ}_{total} ∝ {\sum }_{i} - ln \frac{{f}_{i}}{{f}_{i , ref}} .$$

In one aspect, the analysis comprises a combination of sequence-stability data and consensus analysis of multiple sequence alignment (MSA) of folded versus unfolded proteins.

The disclosure also provides a method of identifying stability-associated peptide fragments, comprising: selecting crossover locations in a set, P, of parental polynucleotides encoding polypeptides that are evolutionary, structurally or evolutionary and structurally related, wherein the set of crossover locations defines N oligonucleotide segments each segment encoding a peptide; performing recombination between a subset, xP^{N}, of the parental polynucleotides having crossover locations to obtain a sample set of recombined, recombinant proteins comprising peptide segments encoded by the oligonucleotide segments, wherein x<1; measuring stability of the sample set of expressed folded recombined, recombinant proteins; performing regression analysis and/or consensus analysis of recombined, recombinant proteins having stability to identify stability-associated peptide segments and the encoding oligonucleotide segment; outputting sequence data and stability measurements for stability-associated peptide segments to a database, wherein the database comprises both nucleotide and amino acid sequences.

Also provided by the disclosure is a database of stability-associated peptide segments with stability values obtained from the method of the disclosure for members of a related family.

The method also includes computer implemented process of the foregoing methods. In one aspect, the computer implemented method includes robotic systems for the generation and/or testing of recombined proteins. For example, in one aspect, the disclosure provides a computer implemented method comprising: selecting crossover locations in a set, P, of parental polynucleotides encoding polypeptides that are evolutionary, structurally or evolutionary and structurally related, wherein the set of crossover locations defines N oligonucleotide segments each segment encoding a peptide; performing recombination between a subset, xP^{N}, of the parental polynucleotides having crossover locations to obtain a sample set of recombined, recombinant proteins comprising peptide segments encoded by the oligonucleotide segments, wherein x<1; obtaining data from stability measurements of expressed recombined, recombinant proteins in the sample set; performing regression analysis and/or consensus analysis of recombined, recombinant proteins having stability to identify stability-associated peptide segments and the encoding oligonucleotide segment; generating a stabilized polypeptide encoded by a combination of oligonucleotide encoding stability-associated peptide segments; and outputting the sequence of the stabilized polypeptide to a user.

Other aspects will be apparent from the following detailed description, figures and claims.

### BRIEF DESCRIPTION OF THE DRAWING

FIG. 1A-C show thermostabilities of parental and chimeric cytochromes P450 vary widely and are predicted by an additive model. a, The distribution of T₅₀ values for 184 chimeric cytochromes P450 are shown, with T₅₀s for parents A1, A2 and A3 indicated (solid lines), including four experimental replicate measurements for A2 to examine measurement variability (dotted lines, standard deviation of 1.0° C.). Some chimeras are more stable than the most stable parent. b, Predicted T₅₀ from a simple linear model correlates with the measured T₅₀ for 184 P450 chimeras, with r=0.856. c, Linear model derived from data in b accurately predicts stabilities of 20 new chimeras, including the most-thermostable P450 (MTP) (top rightmost point).

FIG. 2A-B show relative chimera thermostabilities and folding status can be predicted from sequence element frequencies in a multiple sequence alignment of folded proteins. a, Consensus energies computed from fragment frequencies of folded chimeras correlate with measured thermostabilities (T₅₀s) of 204 chimeric proteins. b, The distribution of consensus energies of 613 folded chimeras and 334 unfolded chimeras (minus chimeras having A2 at position 4). Folded chimeras (dark grey) have lower consensus energies than unfolded chimeras (light grey).

FIG. 3A-B show data training and test of linear regression analysis. a. Predicted T₅₀ compared to experimental T₅₀ for the training data set. The r value for the regression line is 0.892. Squares represent outlier points removed after training. b. Predicted T₅₀ using the regression model parameter from the training in (a) compared to measured T₅₀ for the test data set. The r value for the regression line is 0.857.

FIG. 4 shows prediction accuracy (indicated by correlation coefficient between predicted T₅₀ and measured T₅₀) is related to the number of chimeras used for regression analysis.

FIG. 5 shows prediction of T₅₀s of 6,561 members of the P450 SCHEMA library using the linear regression model parameters obtained from the 204 T₅₀ measurements (Table 4).

FIG. 6 shows prediction accuracy (indicated by the Spearman rank-order correlation coefficient between predicted consensus energies and measured T₅₀) is related to the number of chimeras used for consensus analysis.

FIG. 7A-B shows sequence diversity for 44 stable chimeric cytochrome P450 heme domains and the three parent sequences. a. The number of amino acid differences between each pair of chimeras (black) and for parent-chimera pairs (grey). Pairwise sequence differences (excluding parent-parent pairs) range from 7 to 146 amino acids. b. It is not possible to create a two-dimensional illustration with all chimera-chimera Euclidean distances perfectly proportional to the underlying sequence differences. Multi-dimensional scaling in XGOBI (D F Swayne, D Cook, and A Buja, J. Comp. Graph. Stat. (1998), 7, 113-30) was used to optimize a two-dimensional representation that minimizes the discrepancy between the Euclidean distances and the sequence differences.

FIG. 8 shows a comparison of the ranking performance using regression (circles) to the ranking performance using consensus (filled circles). The points represent the performance of each ranking method when partitioning the set of three parents and 205 chimeras with measured T₅₀ values into the top 10, 20, 30 . . . 200. For example, the y-positions of the leftmost points indicate that the consensus method correctly flags 3 of the top 10 chimeras while the regression method correctly flags 6. The x-positions of the leftmost points indicate that the consensus method correctly flags 191 of the bottom 198 chimeras while the regression method correctly flags 194. The regression model has superior ranking performance for all threshold choices.

### DETAILED DESCRIPTION

As used herein and in the appended claims, the singular forms “a,” “and,” and “the” include plural referents unless the context clearly dictates otherwise. Thus, for example, reference to “a domain” includes a plurality of such domains and reference to “the protein” includes reference to one or more proteins, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

An “amino acid” is a molecule having the structure wherein a central carbon atom (the -carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

“Protein” or “polypeptide” refers to any polymer of two or more individual amino acids (whether or not naturally occurring) linked via a peptide bond, and occurs when the carboxyl carbon atom of the carboxylic acid group bonded to the -carbon of one amino acid (or amino acid residue) becomes covalently bound to the amino nitrogen atom of amino group bonded to the -carbon of an adjacent amino acid. The term “protein” is understood to include the terms “polypeptide” and “peptide” (which, at times may be used interchangeably herein) within its meaning. In addition, proteins comprising multiple polypeptide subunits (e.g., DNA polymerase III, RNA polymerase II) or other components (for example, an RNA molecule, as occurs in telomerase) will also be understood to be included within the meaning of “protein” as used herein. Similarly, fragments of proteins and polypeptides are also within the scope of the invention and may be referred to herein as “proteins.” In one aspect of the disclosure, a stabilized protein comprises a chimera of two or more parental peptide segments.

A “peptide segment” refers to a portion or fragment of a larger polypeptide or protein. A peptide segment need not on its own have functional activity, although in some instances, a peptide segment may correspond to a domain of a polypeptide wherein the domain has its own biological activity. A stability-associated peptide segment is a peptide segment found in a polypeptide that promotes stability, function, or folding compared to a related polypeptide lacking the peptide segment. A destabilizing-associated peptide segment is a peptide segment that is identified as causing a loss of stability, function or folding when present in a polypeptide.

A particular amino acid sequence of a given protein (i.e., the polypeptide's “primary structure,” when written from the amino-terminus to carboxy-terminus) is determined by the nucleotide sequence of the coding portion of a mRNA, which is in turn specified by genetic information, typically genomic DNA (including organelle DNA, e.g., mitochondrial or chloroplast DNA). Thus, determining the sequence of a gene assists in predicting the primary sequence of a corresponding polypeptide and more particular the role or activity of the polypeptide or proteins encoded by that gene or polynucleotide sequence.

“Polynucleotide” or “nucleic acid sequence” refers to a polymeric form of nucleotides. In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. A polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term polynucleotide encompasses genomic DNA or RNA (depending upon the organism, i.e., RNA genome of viruses), as well as mRNA encoded by the genomic DNA, and cDNA.

A “nucleic acid segment,” “oligonucleotide segment” or “polynucleotide segment” refers to a portion of a larger polynucleotide molecule. The polynucleotide segment need not correspond to an encoded functional domain of a protein; however, in some instances the segment will encode a functional domain of a protein. A polynucleotide segment can be about 6 nucleotides or more in length (e.g., 6-20, 20-50, 50-100, 100-200, 200-300, 300-400 or more nucleotides in length). A stability-associated peptide segment can be encoded by a stability-associated polynucleotide segment, wherein the peptide segment promotes stability, function, or folding compared to a polypeptide lacking the peptide segment.

A chimera is a combination of at least two segments of at least two different parent proteins. As appreciated by one of skill in the art, the segments need not actually come from each of the parents, as it is the particular sequence that is relevant, and not the physical nucleic acids themselves. For example, a chimeric P450 will have at least two segments from two different parent P450s. The two segments are connected so as to result in a new P450. In other words, a protein will not be a chimera if it has the identical sequence of either one of the parents. A chimeric protein can comprise more than two segments from two different parent proteins. For example, there may be 2, 3, 4, 5-10, 10-20, or more parents for each final chimera or library of chimeras. The segment of each parent enzyme can be very short or very long, the segments can range in length of contiguous amino acids from 1 to the entire length of the protein. In one embodiment, the minimum length is 10 amino acids. In one embodiment, a single crossover point is defined for two parents. The crossover location defines where one parent's amino acid segment will stop and where the next parent's amino acid segment will start. Thus, a simple chimera would only have one crossover location where the segment before that crossover location would belong to one parent and the segment after that crossover location would belong to the second parent. In one embodiment, the chimera has more than one crossover location. For example, 2, 3, 4, 5, 6, 7, 8, 9, 10, 11-30, or more crossover locations. How these crossover locations are named and defined are both discussed below. In an embodiment where there are two crossover locations and two parents, there will be a first contiguous segment from a first parent, followed by a second contiguous segment from a second parent, followed by a third contiguous segment from the first parent. Contiguous is meant to denote that there is nothing of significance interrupting the segments. These contiguous segments are connected to form a contiguous amino acid sequence. For example, a P450 chimera from CYP102A1 (hereinafter “A1”) and CYP102A2 (hereinafter “A2”), with two crossovers at 100 and 150, could have the first 100 amino acids from A1, followed by the next 50 from A2, followed by the remainder of the amino acids from A1, all connected in one contiguous amino acid chain. Alternatively, the P450 chimera could have the first 100 amino acids from A2, the next 50 from A1 and the remainder followed by A2. As appreciated by one of skill in the art, variants of chimeras exist as well as the exact sequences. Thus, not 100% of each segment need be present in the final chimera if it is a variant chimera. The amount that may be altered, either through additional residues or removal or alteration of residues will be defined as the term variant is defined. Of course, as understood by one of skill in the art, the above discussion applies not only to amino acids but also nucleic acids which encode for the amino acids.

Protein stability is a key factor for industrial protein use (e.g., enzyme reaction) in denaturing conditions required for efficient product development and in therapeutic and diagnostic protein products. Methods for optimizing protein stability have included directed evolution and domain shuffling. However, screening and developing such recombinant libraries is difficult and time consuming.

Directed evolution has proven to be an effective technique for engineering proteins with desired properties. Because the probability of a protein retaining its fold and function decreases exponentially with the number of random substitutions introduced (Bloom et al., Proc. Natl. Acad. Sci. USA, 102, 606-611, 2005), only a few mutations are made in each generation in order to maintain a reasonable fraction of functional proteins for screening (Voigt et al., Advances in Protein Chemistry, Vol 55, Academic Press, pp. 79-160, 2001). Creating libraries with higher levels of mutation while maintaining structure and function requires identifying mutations that are less likely to disrupt the structure (Lutz and Patrick, Curr. Opin. Biotechnol., 15, 291-297, 2004). One strategy to accomplish this is homologous recombination: mutations introduced by recombination are less deleterious than random mutations because they are compatible with the backbone structure (Drummond et al., Proc. Natl. Acad. Sci. USA, 102, 5280-5385, 2005). Random recombination of highly similar proteins often generates libraries with a high fraction of functional sequences; however, as more distantly related proteins are recombined, the fraction of chimeric proteins that fold correctly decreases.

Efforts have been made to identify consensus mutations that provide stabilizing effects. Consensus stabilization has been shown to be effective in some cases and to some degree, but not all consensus mutations are stabilizing (e.g., more than 40% of the consensus residues identified from multiple sequence alignment of naturally occurring β-lactamases are in fact destabilizing rather than stabilizing (Amin et al. Prot. Eng. Des. & Sel., 17(11):787-793, 2004)). These methods have two problems: first single mutations generally have small effects on stability and second not all mutations can be combined such that the stabilizing effects can be properly measured.

Thus, methods of protein development have focused on providing stabilized proteins by generating a large number of recombined proteins and assaying each recombined protein for activity. A method of identifying stabilizing mutations is a first step in removing or narrowing possible candidates. For this reason it is of value to be able to make multiple versions of a protein that are stabilized. If one has many stable variants to choose from, then those variants that exhibit all of the properties of interest can be identified by appropriate analysis of those properties. The disclosure provides a method for making many (e.g., from 1 to many thousand) variants of a protein having amino acid sequences that may differ at multiple amino acid positions and that are stabilized and thus are likely to be functional. Such techniques for generating libraries of stabilized proteins have not previously been provided in the art.

A number of techniques are used for generating novel proteins including, for example, rational design, which uses computational methods to identify sites for introducing disulfide bonds; directed evolution; and consensus stabilization. The foregoing methods do not utilize a linear regression or consensus analysis to assist selectively designing stabilized proteins.

Recombination has been widely applied to accelerate in vitro protein evolution. In this process, the genetic information of several genes is exchanged to produce a library of recombined, recombinant mutants. These mutants are screened for improvement in properties of interest, such as stability, activity, or altered substrate specificity. In vitro recombination methods include DNA shuffling, random-priming recombination, and the staggered extension process (StEP). In DNA shuffling, the parental DNA is enzymatically digested into fragments. The fragments can be reassembled into offspring genes. In the random-priming method, template DNA sequences are primed with random-sequence primers and then extended by DNA polymerase to create fragments. The template is removed and the fragments are reassembled into full-length genes, as in the final step of DNA shuffling. In each of these methods, the number of cut points can be increased by starting with smaller fragments or by limiting the extension reaction. StEP recombination differs from the first two methods because it does not use gene fragments. The template genes are primed and extended before denaturation and reannealing. As the fragments grow, they reanneal to new templates and thus combine information from multiple parents. This process is cycled hundreds of times until a full-length offspring gene is formed. The foregoing methods are known in the art.

Recently, it has been shown that recombining genes that have evolved independently in nature is a powerful way to quickly accumulate large improvements in stability and function. Given the explosive growth in the gene databases due to the exhaustive sequencing of large numbers of organisms, the sequences of homologous genes are easily accessible. These sequences can be synthesized or cloned for evolution of protein functions by recombination methods described above and known in the art.

Common to these experimental approaches to recombination in vitro is that the genes are cut and reformed randomly, that is, there is little or no a priori input into the experimental protocol regarding which genes are chosen for recombination and where the cut points should occur, other than in regions of high sequence similarity. Using the SCHEMA method (described further herein) sequences are predicted that are more likely to generate diverse recombined, recombinant gene libraries and the desired improvements in the recombined, recombinant genes.

As a first step in performing any recombination techniques a set of related polypeptides is identified. The relatedness of the polypeptides can be determined in any number of ways known in the art. For example, polypeptides may be related structurally either in their primary sequence or in the secondary or tertiary sequence. Methods of identifying sequence identity or 3D structural similarities are known and are further described herein. Another method to identify a related polypeptide is through evolutionary analysis. Evolutionary trees have been developed for a large number of proteins and are available to those of skill in the art.

A parental sequence used as a basis for defining a set of related polypeptides can be provided by any of a number of mechanisms, including, but not limited to, sequencing, or querying a nucleic acid or protein database. Additionally, while the parental sequence can be provided in a physical sense (e.g., isolated or synthesized), typically the parental sequence or sequences are obtain in silico.

For embodiments of the disclosure involving amino acid sequences, the parental sequences typically are derived from a common family of proteins having similar three-dimensional structures (e.g., protein superfamilies). However, the nucleic acid sequences encoding these proteins might or might not share a high degree of sequence identity. As described later herein, the methods include assessing crossover positions using any number of techniques (e.g., SCHEMA etc.).

Sequence similarity/identity of various stringency and length can be detected and recognized using a number of methods or algorithms known to one of skill in the art. For example, many identity or similarity determination methods have been designed for comparative analysis of sequences of biopolymers, for spell-checking in word processing, and for data retrieval from various databases. With an understanding of double-helix pair-wise complement interactions among the four principal nucleobases in natural polynucleotides, models that simulate annealing of complementary homologous polynucleotide strings can also be used as a foundation of sequence alignment or other operations typically performed on the character strings corresponding to the sequences herein (e.g., word-processing manipulations, construction of figures comprising sequence or subsequence character strings, output tables, etc.). An example of a software package for calculating sequence identity is BLAST, which can be adapted to the disclosure by inputting character strings corresponding to the sequences herein.

After providing parental sequences, the sequences are aligned. In other embodiments, a plurality of parental sequences are provided, which are then aligned with either a reference sequence, or with one another. Alignment and comparison of relatively short amino acid sequences (for example, less than about 30 residues) is typically straightforward. Comparison of longer sequences can require more sophisticated methods to achieve optimal alignment of two sequences.

Optimal alignment of sequences can be performed, for example, by a number of available algorithms, including, but not limited to, the “local homology” algorithm of Smith and Waterman (Adv. Appl. Math. 2:482, 1981), the “homology alignment” algorithm of Needleman and Wunsch (J. Mol. Biol. 48:443, 1970), the “search for similarity” method of Pearson and Lipman (Proc. Natl. Acad. Sci. USA 85:2444, 1988), or by computerized implementations of these algorithms (e.g., GAP, BESTFIT, FASTA and TFASTA available in the Wisconsin Genetics Software Package Release 7.0, Genetics Computer Group, 575 Science Dr., Madison, Wis.; and BLAST, see, e.g., Altschul et al., Nuc. Acids Res. 25:3389-3402, 1977 and Altschul et al., J. Mol. Biol. 215:403-410, 1990). Alternatively, the sequences can be aligned by inspection. Generally the best alignment (i.e., the relative positioning resulting in the highest percentage of sequence identity over the comparison window) generated by the various methods is selected. However, in certain embodiments of the disclosure, the best alignment may alternatively be a superpositioning of selected structural features, and not necessarily the highest sequence identity.

The term “sequence identity” means that two amino acid sequences are substantially identical (i.e., on an amino acid-by-amino acid basis) over a window of comparison. The term “sequence similarity” refers to similar amino acids that share the same biophysical characteristics. The term “percentage of sequence identity” or “percentage of sequence similarity” is calculated by comparing two optimally aligned sequences over the window of comparison, determining the number of positions at which the identical residues (or similar residues) occur in both polypeptide sequences to yield the number of matched positions, dividing the number of matched positions by the total number of positions in the window of comparison (i.e., the window size), and multiplying the result by 100 to yield the percentage of sequence identity (or percentage of sequence similarity). With regard to polynucleotide sequences, the terms sequence identity and sequence similarity have comparable meaning as described for protein sequences, with the term “percentage of sequence identity” indicating that two polynucleotide sequences are identical (on a nucleotide-by-nucleotide basis) over a window of comparison. As such, a percentage of polynucleotide sequence identity (or percentage of polynucleotide sequence similarity, e.g., for silent substitutions or other substitutions, based upon the analysis algorithm) also can be calculated. Maximum correspondence can be determined by using one of the sequence algorithms described herein (or other algorithms available to those of ordinary skill in the art) or by visual inspection.

As applied to polypeptides, the term substantial identity or substantial similarity means that two peptide sequences, when optimally aligned, such as by the programs BLAST, GAP or BESTFIT using default gap weights or by visual inspection, share sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described in detail below) or by visual inspection, share sequence identity or sequence similarity.

One example of an algorithm that is suitable for determining percent sequence identity or sequence similarity is the FASTA algorithm, which is described in Pearson, W. R. & Lipman, D. J., (1988) Proc. Natl. Acad. Sci. USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: -5, k-tuple=2; joining penalty=40, optimization=28; gap penalty −12, gap length penalty=−2; and width=16.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395).

Another example of an algorithm that is suitable for multiple DNA and amino acid sequence alignments is the CLUSTALW program (Thompson, J. D. et al., (1994) Nuc. Acids Res. 22:4673-4680). CLUSTALW performs multiple pairwise comparisons between groups of sequences and assembles them into a multiple alignment based on sequence identity. Gap open and Gap extension penalties were 10 and 0.05 respectively. For amino acid alignments, the BLOSUM algorithm can be used as a protein weight matrix (Henikoff and Henikoff, (1992) Proc. Natl. Acad. Sci. USA 89:10915-10919).

Another method of determining relatedness is through protein and polynucleotide alignments. Common methods include using sequence based searches available on-line and through various software distribution routes. Homology or identity at the amino acid or nucleotide level can be determined by BLAST (Basic Local Alignment Search Tool) and by ClustalW analysis using the algorithm employed by the programs blastp, blastn, blastx, tblastn and tblastx (Karlin et al., Proc. Natl. Acad. Sci. USA 87, 2264-2268, 1990; Thompson et al., Nucleic Acids Res 22, 4673-4680, 1994; and Altschul, J. Mol. Evol. 36, 290-300, 1993, (fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases (see Altschul et al., Nature Genetics 6, 119-129, 1994, which is fully incorporated by reference). The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc. Natl. Acad. Sci. USA 89, 10915-10919, 1992, fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively.

Accordingly, by using such methods families or groups of structurally related polypeptides can be identified. Typically the protein homology (whether they are evolutionarily, and therefore structurally, related) is determined primarily by sequence similarity (sequences are more similar than expected at random). Sequences that are as low as 15-20% similar by alignments are likely related and encode proteins with similar structures. Additional structural relatedness can be determine using any number of further techniques including, but not limited to, X-ray crystallography, NMR, searching a protein structure databases, homology modeling, de novo protein folding, and computational protein structure prediction. Such additional techniques can be used alone or in addition to sequence-based alignment techniques. In one aspect, the degree of similarity/identity between two proteins or polynucleotide sequences should be at least about 20% or more (e.g., 30%, 35%, 40%, 45%, 50%, 55%, 60%, 65%, 70%, 75%, 80%, 85%, 90%, 95%, 98% or 99%).

In some aspects, parent sequences are chosen from a database of sequences, by a sequence homology search such as BLAST. Parental sequences will typically be between about 20% and 95% identical, typically between 35 and 80% identical. The lower the identity, the more the mutation level (and possibly the greater the possible stability enhancement and functional variation in the resulting sequences) following recombination between parental strands. The higher the identity, the higher the probability the sequences will fold and function.

If polypeptides sequences are used to identify structurally, evolutionary or structural and evolutionary related proteins, one can identify the corresponding polynucleotides sequences through databases available to the public including GenBank and NCBI. The polynucleotide sequences will be used to identify crossover locations for recombination using, for example, SCHEMA methods described herein. If the polynucleotides sequence is used to identify structural and evolutionarily related proteins, the corresponding polypeptide sequences can be identified through databases available to the public. In one aspect of the disclosure both the polynucleotide and polypeptide sequences are used, however, it will be recognized that the polynucleotide sequence alone can be used in the methods of the disclosure.

In addition to computer algorithms and visual alignment techniques described above to determine identity or similarity, other techniques can be used. For example, hybridization techniques can be used to identify polynucleotides that are substantially identical. Such techniques are based upon the base pairing of DNA and RNA to complementary strands under various conditions the promote binding. “Stringent conditions” are those that (1) employ low ionic strength and high temperature for washing, for example, 0.5 M sodium phosphate buffer at pH 7.2, 1 mM EDTA at pH 8.0 in 7% SDS at either 65° C. or 55° C., or (2) employ during hybridization a denaturing agent such as formamide, for example, 50% formamide with 0.1% bovine serum albumin, 0.1% Ficoll, 0.1% polyvinylpyrrolidone, 0.05 M sodium phosphate buffer at pH 6.5 with 0.75 M NaCl, 0.075 M sodium citrate at 42° C. Another example is use of 50% formamide, 5×SSC (0.75 M NaCl, 0.075 M sodium citrate), 50 mM sodium phosphate at pH 6.8, 0.1% sodium pyrophosphate, 5×Denhardt's solution, sonicated salmon sperm DNA (50 μg/ml), 0.1% SDS and 10% dextran sulfate at 55° C., with washes at 55° C. in 0.2×SSC and 0.1% SDS. A skilled artisan can readily determine and vary the stringency conditions appropriately to obtain a clear and detectable hybridization signal. Polynucleotides that hybridize to one another share a degree of identity related to the stringency of the conditions used.

Once a set of structurally, evolutionary, or structural and evolutionary polypeptides have been identified and the corresponding polynucleotide sequences identified, the sequence are analyzed for crossover locations. The term “crossover location” as used herein refers to a position in a sequence at which the origin of that portion of the sequence changes, or “crosses over” from one source to another (e.g., a terminus of a subsequence involved in an exchange between parental sequences).

After identifying the parental sequences (e.g., the first sequence, second sequences, and optional additional sequences), portions of the parental sequences are replaced, swapped or exchanged. Each exchange occurs between first and second crossover locations on the two parental sequences encompassing the selected segments (subsequence of amino acids or nucleotides) of a given exchange. Optionally, multiple segments can be swapped at a plurality of crossover positions in a given parental sequence, thereby generating a chimeric polypeptide having more than one segment inserted (from one or more parental sequences). With reference to a nucleic acid, the crossover sites define the 5′ and 3′ ends of the regions of exchanged oligonucleotides (e.g., the positions at which the recombination occurs). For protein sequences, the crossover sites are defined by the start (N-terminus) and end (C-terminus) of the exchanged amino acid residues. In some embodiments, the first crossover site coincides with the 5′ end of the nucleic acid, or the N-terminus of the amino acid sequence. In other embodiments, the second crossover site coincides with the 3′ end of the nucleic acid, or the C-terminus of the amino acid sequence. The length of the selected segment to be exchanged will vary.

Selection of crossover sites can be performed empirically (e.g., starting at every fifth element in the sequence) or the selection can be based upon additional criteria. Considering that co-variation of amino acids during evolution allows proteins to retain a given fold, tertiary structure or function while altering other traits (such as specificity), this information can be useful in selecting possible crossover locations which will not be detrimental to the overall structure or function of the molecule. Alternatively, the regions for exchange can be selected, for example, by targeting a desired activity (e.g., the active site of a protein or catalytic nucleic acid) or specific structural feature (e.g., replacement of alpha helices or strands of a beta sheet). Visual analysis of the alignment of the parent sequence with the contact map and/or tertiary structure of the reference protein can also focus the analytical efforts on regions of structural interest.

The methods of recombining the one or more segments between parental sequences to generate a chimeric polypeptide can be performed in silico. In silico methods of recombination use algorithms on a computer to recombine sequence strings which correspond to homologous (or even non-homologous) nucleic acids. The resulting recombined sequences are optionally converted into chimeric polynucleotides by synthesis, e.g., in concert with oligonucleotide synthesis/gene reassembly techniques. This approach can generate random, partially random or designed variants. Many details regarding in silico recombination, including the use of algorithms, operators and the like in computer systems, combined with generation of corresponding polynucleotides (and/or proteins), as well as combinations of designed polynucleotides and/or proteins (e.g., based on cross-over site selection) are known in the art.

In brief, desirable crossover locations can be selected between two or more sequences, e.g., following an approximate sequence alignment, by performing Markov chain modeling, or any other desired selection method including the SCHEMA method. In this way, it is possible to identify crossover locations, and reduce the total number of bridging oligonucleotides, this time to a number which can actually be synthesized to provide a useful number of bridging oligonucleotides to facilitate recombination of segments. Crossover locations can also be identified by comparing the structures (either from crystals, nmr, dynamic simulations, or any other available method) of proteins corresponding to nucleic acids to be recombined. All possible pairwise combinations of structures can be overlaid. Amino acids can be identified as possible crossover points when they overlap with each other on the parental structures, or when they and their nearest neighbors overlap within similar distance criteria. Bridging oligos can be built for each crossover location. Accordingly, an in silico selection of recombined molecules and the step of cross-over selection in parental sequences are combined into a single simultaneous step.

Crossovers are first determined base on the protein sequence. But for convenience of construction of the new, recombined genes, it is sometimes useful to move the crossover location 1 to 6 base pairs in terms of the polynucleotide sequence based upon the gene recombination methods (e.g., any requirement for different dangling ends of the DNA fragments).

In one aspect, the methods of the disclosure use a SCHEMA algorithm to identify and select crossover locations. The SCHEMA method improves the probability distribution for the cut points, given structural information and the sequences of the parents to be shuffled. This approach can be divided into at least two parts. First, through a sequence alignment of the parents, the number of possible crossover points is reduced by calculating all the possible annealing points based on sequence similarity. This process reduces the search space considerably. Possible crossover points are eliminated based on the crossover disruption associated with each recombined mutant. Crossover disruption is a concept borrowed from genetic algorithm theory, which states that recombination is most successful when the fewest good interactions between amino acids are broken by the crossovers. A good interaction is defined as any coupled contribution between amino acids where the combination of the two amino acids is better that the sum of the individual contributions. Recombining sets of amino acid residues that correspond to clusters of good interactions minimizes the crossover disruption. The offspring genes that are most likely to have the beneficial sets of amino acids from each parent gene, without destabilizing the structure.

For most recombination methods, the crossover points occur in regions where there is adequate DNA sequence similarity to promote reannealing. In one embodiment of the SCHEMA algorithm, the first step is to calculate the possible cut points by enumerating the regions of sequence similarity through a sequence alignment as described above. From this sequence alignment, all the possible crossover points between the parents are calculated, according to some minimum overlap in DNA sequence. In one aspect, for example, the same two amino acids exist in either direction from the cut point on the primary sequence. In other words, the cut point can occur where the recombined sequences share four identical amino acids. Different algorithms can be constructed using DNA sequence similarity, rather than identity, for the cut point criterion and including higher crossover probabilities when the similarity is greater.

A coupling interaction is then defined as any interaction between amino acids. If the property of interest is stability, this includes hydrogen bonds, electrostatic interactions, and Van der Waals interactions. The energy of interaction is calculated for all pairwise combinations of residues using the wild-type conformation of amino acids in the three-dimensional crystal structure. To calculate the interactions, a DREIDING force field, with an additional hydrogen-bonding term used previously in computational protein design is used. If interaction energy between two residues is below a certain cutoff value, the residues are considered to be coupled. For example, a cutoff of −0.25 kcal/mol can be used. The results are robust with respect to the choice of this cutoff. A coupling criterion that the absolute value of the interaction energy be above some threshold is also successful.

The determination of the coupling between residues is not limited to the approach outlined above. Various force fields can be used, including using CHARMM (Brooks et al., 1983) or any generic Van der Waals and electrostatic potential (Hill, 1960). A mean-field approach can also be used to weight the probability of all amino acids existing at each site and the associated energy, thus giving a better estimate of the coupling. In addition, a simple distance measure can be imposed. If two residues are within a certain cutoff distance, then they can be considered as interacting.

An algorithm is used to generate genes by recombining the parents in a way that is consistent with the potential crossover points calculated above. For example, a random parent is chosen, this parent is copied to the offspring until a possible cut point is reached. A random number between 0 and 1 is chosen, and if this number is below a crossover probability p_{c}, then a new parent is randomly chosen and copied to the offspring until a new possible crossover point is reached. This process is repeated until the entire offspring gene is constructed. A further restriction can be imposed where each fragment has to be at least eight amino acids long before another crossover can occur. This restriction can be varied as desired.

The computation can be applied to the different methods through the interpretation of p_{c}, which is directly related to the average fragment size. In the DNAse and restriction enzyme approach to fragmentation, the fragment size is controlled by the concentration of enzyme and other experimental conditions. In the restriction enzyme case, it is also controlled by the diversity of enzymes. As the reaction is run with higher concentrations of enzyme, the size of the fragments gets smaller. Similarly, in the random-priming recombination, the fragment size is controlled by the length of time for which the polymerase is allowed to build the fragments.

Once a recombined polypeptide is generated in silico, its crossover disruption is calculated by counting the number of coupling interactions that are broken by the cut points. To do this, all the interactions are shared between fragments of different parents are summed, while the interactions within fragments and shared between fragments from the same parent are ignored. This can be repeated until sufficient statistics have been accumulated. In practice, between 10⁴ to 10⁶ recombined polypeptides are generated in silico.

Using the foregoing methods comprising identifying a plurality (P) of evolutionary, structurally or evolutionary and structurally related polypeptides and selecting a set of crossover locations comprising N peptide segments, the total number of recombined chimeric polypeptides that can be generated is P^{N}.

A sample set (xP^{N}) of recombined proteins comprising peptide segments from each of the at least first polypeptide and second polypeptide, wherein x<1 is generated by recombinant molecular biology techniques known in the art. The resulting recombined chimeric polypeptides are expressed and assayed. Typically the sample set of expressed polypeptides comprises from about 10-1000 (e.g., 20-200, 30-100) and any range or number there between. For example, x can be a factor of 0.05 to 0.9.

Natural proteins differ from most polymers in that they predominantly populate a single, ordered three-dimensional structure in solution. It has long been recognized that this ordered structure can be transformed to an approximate random chain by changes in temperature, pressure or solvent conditions (Neurath et al., Chem. Rev. 34: 157-265, 1944). The ability to induce protein unfolding, and subsequent refolding, has allowed scientists to analyze the physical chemistry of the folding reaction in vitro (Schellman, Annu. Rev. Biophys. Bio. 16: 115-37, 1987). These investigations have shed light on the kinetics and thermodynamics of conformational changes in proteins and are of biological interest.

The function of a protein is contingent on the stability of its conformation. Consequently, in the field of protein biochemistry, stability measurements are frequently performed to establish a polypeptide as a stably folded protein and to study the physical forces that lead to its folding (Schellman, Annu. Rev. Biophys. Bio. 16: 115-37, 1987). This is of interest in both industry and medical therapeutics to identify proteins having increased stability to improve therapeutic benefit and industrial applications under extreme conditions. Accordingly, developing proteins having increased stability. Despite their utility, stability measurements currently necessitate time-consuming experiments. In proteomic experiments where a large number of polypeptides often need to be analyzed, stability measurements are not practical. Thus, methods of designing proteins having improved stability and/or activity are useful.

Recent studies have demonstrated that hydrogen exchange coupled with electrospray ionization (ESI) mass spectrometry can qualitatively distinguish native-like proteins from unfolded polypeptides in partially purified samples and can be used to study the kinetics and thermodynamics of folding.

Thermodynamic stability is an important biological property that has evolved to an optimal level to fit the functional needs of proteins. Therefore, investigating the stability of proteins is important not only because it affords information about the physical chemistry of folding, but also because it can provide important biological insights. A proper understanding of protein stability is also useful for technological purposes. The ability to rationally make proteins of high stability, low aggregation or low degradation rates will be valuable for a number of applications. For example, proteins that can resist unfolding can be used in industrial processes that require enzyme catalysis at high temperatures (Van den. Burg et al., Proc. Natl. Acad. Sci. U.S.A. 95(5): 2056-60, 1998); and the ability to produce proteins with low degradation rates within the cell can help to maximize production of recombinant proteins (Kwon et al., Protein Eng. 9(12): 1197-202, 1996).

Stability measurements can also be used as probes of other biological phenomena. The most basic of these phenomena is biological activity. The ability of proteins to populate their native states is a universal requirement for function. Therefore, stability can be used as a convenient, first level assay for function. For example, libraries of polypeptide sequences can be tested for stability in order to select for sequences that fold into stable conformations and might potentially be active (Sandberg et al., Biochem. 34: 11970-78, 1995).

Changes in stability can also be used to detect binding. When a ligand binds to the native conformation of a protein, the global stability of a protein is increased Schellman, Biopolymers 14: 999-1018, 1975; Pace & McGrath, (1980) J. Biol. Chem. 255: 3862-65; Pace & Grimsley, Biochem. 27: 3242-46, 1988). The binding constant can be measured by analyzing the extent of the stability increase. This strategy has been used to analyze the binding of ions and small molecules to a number of proteins (Pace & McGrath, (1980) J. Biol. Chem. 255: 3862-65; Pace & Grimsley, (1988) Biochem. 27: 3242-46; Schwartz, (1988) Biochem. 27: 8429-36; Brandts & Lin, (1990) Biochem. 29: 6927-40; Straume & Freire, (1992) Anal. Biochem. 203: 259-68; Graziano et al., (1996) Biochem. 35: 13386-92; Kanaya et al., (1996) J. Biol. Chem. 271: 32729-36).

The linkage between stability and binding has recently been implemented as a method to detect ligand binding (U.S. Pat. No. 5,679,582 to Bowie & Pakula). This method, however, does not take advantage of the high sensitivity available from an analytical technique such as MALDI mass spectrometry, and cannot be employed at the low protein levels that MALDI mass spectrometry can detect. Moreover, proteolytic methods can require additional steps to isolate and analyze proteolytic fragments and cannot be performed in an in vivo setting. Finally, this method cannot be employed to generate quantitative measurements of protein stability.

The expressed chimeric recombinant proteins are measured for stability and/or biological activity. Techniques for measuring stability and activity are known in the art and include, for example, the ability to retain function (e.g. enzymatic activity) at elevated temperature or under ‘harsh’ conditions of pH, salt, organic solvent, and the like; and/or the ability to maintain function for a longer period of time (e.g., in storage in normal conditions, or in harsh conditions). Function will of course depend upon the type of protein being generated and will be based upon its intended purpose. For example, P450 mutants can be tested for the ability to convert alkanes to alcohols under various conditions of pH, solvents and temperature. Other enzyme assays are known in the art for various industrial enzymes selected from the group consisting of carbohydrases, alpha-amylase, β-amylase, cellulase, β-glucanase, β-glucosidase, dextranase, dextrinase, glucoamylase, hemmicellulase/pentosanase/xylanase, invertase, lactase, pectinase, pullulanase, proteases, oxygenases, acid proteinase, alkaline protease, pepsin, peptidases, aminopeptidase, endo-peptidase, subtilisin, lipases and esterases, aminoacylase, glutaminase, lysozyme, penicillin acylase, isomerase, oxireductases, alcohol dehydrogenase, amino acid oxidase, catalase, chloroperoxidase, peroxidase, lyases, acetolactate decarboxylase, aspartic β-decarboxylase, histidase, transferases, and cyclodextrin glycosyltransferase. Stability test can comprise chemical stability measurements, functional stability measurements and thermal stability measurements. Chemical stability measurements comprise chemical denaturation measurements. Thermal stability measurements comprise thermal denaturation measurements. Function stability measurement can comprise ligand or substrate binding techniques. Other techniques can include various electrophoretic techniques, spectroscopy and the like.

In one aspect, folded proteins are used in the analysis. In another aspect, only proteins that are sufficiently expressed are analyzed. Which proteins these are depends on how one measures stability (e.g., if it is by activity loss, then there should enough activity produced in order to measure a loss). If stability is measured by purifying the protein, then there should be enough folded protein to purify. Accordingly, the recombinant chimeric protein should be expressed and its stability measurable, quantitatively, in order for it to be analyzed.

The disclosure shows that chimeric proteins exhibit a broad range of stabilities, and that stability of a given folded sequence can be predicted based on data (either stability or folding status) from a limited sampling of the chimeric library and that further development and design can be optimized using a regression model of analysis of stabilized proteins.

Recombinant chimeric proteins that demonstrate stability are analyzed to determine their chimeric components. The regression analysis comprises determining sequence-stability data and the consensus analysis comprises determining multiple sequence alignment (MSA) of folded versus unfolded proteins.

The disclosure includes methods of identifying and generating stable proteins comprising recombination of evolutionary, structurally or evolutionary and structurally related polypeptide through a process of recombination, consensus analysis and/or linear regression analysis of recombined chimeric proteins to identify peptide segments that improve protein stability. For example, a population of P parental proteins having N crossover fragments would generated a recombinant library population of P^{N} members. A method of the disclosure uses recombination, a SCHEMA method and regression analysis to reduce the number of members needed to be generated as well as predicting and designing polypeptides having increased stability and/or activity. In one aspect, the regression comprises sequence-stability data. In another aspect, the regression analysis is based on consensus analysis of the multiple sequence alignment.

For example, in one aspect, the regression analysis comprises a linear model. In one aspect,

$${T}_{50} = {a}_{0} + {\sum }_{i} {\sum }_{j} {a}_{ij} {x}_{ij}$$

was used for regression, where T₅₀ is the dependent variable and fragments xᵢⱼ (from the i^{th} position and j^{th} parent, where, e.g., i=1, and j=2 or 3) are the independent variables. The xᵤ are dummy-coded, such that if a chimera has fragment 1 from parent 2, x₁₂=1 and x₁₃=0. Using this calculation a reference polypeptide comprising known sequence, stability and/or function, was used for all eight positions, so the constant term (a₀) is the predicted T₅₀ of the parent and the regression coefficients aᵢⱼ represent the thermostability contributions of fragments xᵤ relative to the corresponding reference polypeptide fragments. In general, the reference fragment at each of the 8 positions can be chosen arbitrarily. Regression was performed using SPSS(SPSS for Windows, Rerl. 11.0.1. 2001. Chicago: SPSS Inc.).

In yet another aspect, a consensus energy calculation is used to identify stability conferring fragments. The linear regression model uses fewer measurements and provides more true positives with fewer false positives than the consensus approach based on folding status.

Consensus stabilization is based on the idea that the frequencies of sequence elements correlate with their corresponding stability contributions. This correlation is typically assumed to follow a Boltzmann-like exponential relationship. Such a relationship is most sensible if, in analogy to statistical mechanics, the sequences are randomly sampled from the ensemble of all possible folded proteins (e.g., P450s). Natural sequences are related by divergent evolution and may not comprise such a sample. A chimeric protein data set, in contrast, represents a large and nearly random sample of all possible chimeras. The data provided herein supports the underlying consensus stabilization approaches: sequence elements contribute additively to stability, stabilizing fragments occur at higher frequencies among folded sequences, and the consensus sequence is the most stable in the ensemble. These results demonstrate the tolerance of the consensus stabilization idea to different ensembles (chimeric libraries versus evolved families) and sequence changes (recombination versus stepwise mutation). Unlike previous implementations of consensus stabilization, however, the approach described here generates dozens of stable proteins, and these proteins differ from each other and from the parents at many amino acid residues.

In this aspect, assuming the frequency of a fragment at position i is exponentially related to its stability contribution and that these fragment contributions are additive, total chimera consensus energy relative to a reference sequence can be calculated from

$${Δɛ}_{total} ∝ {\sum }_{i} - ln \frac{{f}_{i}}{{f}_{i , ref}} ,$$

where f_{Yef} is the ensemble frequency of the fragment at i in a reference sequence. A parental protein with a known stability and sequence was again used as the reference, so that the consensus energy of the parental reference was zero; the choice of reference sequence is arbitrary and does not influence the results. Note that the values reported are actually proportional to energy differences from the reference; referred to as consensus energies for brevity. The raw frequencies fᵢⱼ^{raw} of fragment i from parent j in the folded ensemble may reflect biases in the assembly of chimeras from their constituent fragments. Bias can be assessed by measuring the frequencies fᵢⱼ^{unselected} in an unselected set of sequences to determine the biases bᵢⱼ=nₚₐᵣₑₙₜₛfᵢⱼ^{unselected}, which in an unbiased ensemble will be equal to 1. For the P450 ensemble the fᵢⱼ^{unselected} are known (Table 5). Construction bias can be corrected directly by dividing the fᵢⱼ^{raw} by the bᵢⱼ, and bias-corrected frequencies were used in all analyses.

The high degree of additivity observed are surprising, considering the cooperative nature of protein folding and the many tertiary contacts in the native structure. The additivity of stability changes to proteins has been shown. Non-additive effects are expected when sequence changes are coupled or result in significant structural changes. Structural disruption is less likely in chimeras than with random mutants because all sequence elements are believed to fold to a similar structure in at least one context, that of the parental sequence. Furthermore, such block-additivity can be maximized by the library design, which reduces coupling. SCHEMA (as described above) identifies sequence fragments that minimize the number of contacts, or interactions that can be broken upon recombination. Two residues in a chimera are defined to have a contact if any heavy atoms are within 4.5 Å; the contact is broken if they do not appear together in any parent at the same positions. Among a total of about 500 contacts for a P450 chimera, an average of fewer than 30 were broken for the sequences in the SCHEMA library. The SCHEMA fragments that were swapped in the library have many intra-fragment contacts; the inter-fragment contacts are either few or are conserved among the parents. As a result, the fragments function as pseudo-independent structural modules that make roughly additive contributions to stability. The additivity was strong enough to enable detection of sequencing errors based on deviations from additivity, prediction of thermostabilities for uncharacterized chimeras with high accuracy, and prediction of the T50 of the most stable chimera to within measurement error. Because SCHEMA effectively identifies functional chimeras with other protein scaffolds, such as β-lactamases, this approach allows one to identify novel stable, functional sequences for other protein families.

The methods of the disclosure demonstrated here identify highly stable sequences; recombination ensures that they also retain biological function and exhibit high sequence diversity by conserving important functional residues while exchanging tolerant ones. This sequence diversity can give rise to useful functional diversity. This study demonstrated improvements in activity (on 2-phenoxyethanol) as well as acquisition of entirely new activities (on verapamil and astemizole) in the stabilized P450 enzymes. That the P450 chimeras can produce authentic human metabolites of drugs opens the door to rapid drug metabolic profiling and lead diversification using soluble enzymes that are produced efficiently in E. coli.

Using the methods described herein, novel stabilized proteins can be designed based upon identified stability components. The information related to each stability component (e.g., a stabilized-peptide segment sequence or its corresponding coding sequence) can be identified and stored in a database in order to generated a database of stable peptide sequence components.

The methods of the disclosure provide techniques for identifying stable proteins and structures through reduced library development and screening. Stable proteins developed and identified by the methods of the disclosure are, for example, more robust to random mutations and are often better starting points for engineering to enhance other properties including desired activities.

Although the specific examples provided herein look at cytochrome P450 enzymes, it will be apparent to those of skill in the art, that the methods and techniques described herein are not limited to any one protein family or group.

All classes of molecules and compounds that are utilized in both established and emerging chemical, pharmaceutical, textile, food and feed, detergent markets must meet stringent economical and environmental standards. The synthesis of polymers, pharmaceuticals, natural products and agrochemicals is often hampered by expensive processes which produce harmful byproducts and which suffer from poor or inefficient catalysis. Enzymes, for example, have a number of remarkable advantages which can overcome these problems in catalysis: they act on single functional groups, they distinguish between similar functional groups on a single molecule, and they distinguish between enantiomers. Moreover, they are biodegradable and function at very low mole fractions in reaction mixtures. Because of their chemo-, regio- and stereospecificity, enzymes present a unique opportunity to optimally achieve desired selective transformations. These are often extremely difficult to duplicate chemically, especially in single-step reactions. The elimination of the need for protection groups, selectivity, the ability to carry out multi-step transformations in a single reaction vessel, along with the concomitant reduction in environmental burden, has led to the increased demand for enzymes in chemical and pharmaceutical industries. Enzyme-based processes have been gradually replacing many conventional chemical-based methods. A current limitation to more widespread industrial use is primarily due to the relatively small number of commercially available enzymes. Only .about.300 enzymes (excluding DNA modifying enzymes) are at present commercially available from the >3000 non DNA-modifying enzyme activities thus far described.

The use of enzymes for technological applications also may require performance under demanding industrial conditions. This includes activities in environments or on substrates for which the currently known arsenal of enzymes was not evolutionarily selected. However, the natural environment provides extreme conditions including, for example, extremes in temperature and pH. A number of organisms have adapted to these conditions due in part to selection for polypeptides than can withstand these extremes. In addition, the methods of the disclosure allow for the development and selection of proteins (including enzymes) that have improved stability under these conditions.

In addition to the need for new enzymes for industrial use, there has been a dramatic increase in the need for bioactive compounds with novel activities. This demand has arisen largely from changes in worldwide demographics coupled with the clear and increasing trend in the number of pathogenic organisms that are resistant to currently available antibiotics. For example, while there has been a surge in demand for antibacterial drugs in emerging nations with young populations, countries with aging populations, such as the U.S., require a growing repertoire of drugs against cancer, diabetes, arthritis and other debilitating conditions. The death rate from infectious diseases has increased 58% between 1980 and 1992 and it has been estimated that the emergence of antibiotic resistant microbes has added in excess of $30 billion annually to the cost of health care in the U.S. alone.

The methods of the disclosure are applicable to a wide range of proteins. This method can be applied to improving the stability of industrial enzymes (e.g. those used in bioenergy applications such as cellulases, amylases, and xylanases; those in paper and pulping such as xylanases and laccases; those used in detergents such as proteases and lipases; those used in foods; those used in making chemicals such as lipases and other hydrolases, oxidoreductases). It can also be used to improve stability of therapeutic proteins, proteins used in sensors and diagnostics, and proteins used in other applications. The method can be applied to any protein or protein domain comprising about 50 amino acids or more (e.g., 50-100, 100-200, 200-300, 300-400, 500-1000 or more than 1000 amino acids). Smaller domains or peptide segments generally form part of a larger multi-domain protein (such as the P450 BM3, which is a protein with four ‘domains’). Other protein enzymes that can be designed by the methods of the disclosure comprise industrial enzyme is selected from the group consisting of carbohydrases, alpha-amylase, β-amylase, cellulase, β-glucanase, β-glucosidase, dextranase, dextrinase, glucoamylase, hemmicellulase/pentosanase/xylanase, invertase, lactase, pectinase, pullulanase, proteases, oxygenases, acid proteinase, alkaline protease, pepsin, peptidases, aminopeptidase, endo-peptidase, subtilisin, lipases and esterases, aminoacylase, glutaminase, lysozyme, penicillin acylase, isomerase, oxireductases, alcohol dehydrogenase, amino acid oxidase, catalase, chloroperoxidase, peroxidase, lyases, acetolactate decarboxylase, aspartic β-decarboxylase, histidase, transferases, and cyclodextrin glycosyltransferase. In specific examples provided herein, the disclosure demonstrates that ability identify and develop stabilized P450's (e.g., cytochrome P450's oxygenases).

In another embodiment, the methods and compositions of the disclosure provide for the ability to design lead drug compounds present in an environmental sample. The methods of the invention provide the ability to mine the environment for novel drugs or identify related drugs contained in different microorganisms to generate stable chimeric proteins.

Polyketide synthases enzymes can be designed for improved stability using the methods of the disclosure. Polyketides are molecules which are an extremely rich source of bioactivities, including antibiotics (such as tetracyclines and erythromycin), anti-cancer agents (daunomycin), immunosuppressants (FK506 and rapamycin), and veterinary products (monensin). Many polyketides (produced by polyketide synthases) are valuable as therapeutic agents. Polyketide synthases are multifunctional enzymes that catalyze the biosynthesis of a huge variety of carbon chains differing in length and patterns of functionality and cyclization. Polyketide synthase genes fall into gene clusters and at least one type (designated type I) of polyketide synthases have large size genes and enzymes, complicating genetic manipulation and in vitro studies of these genes/proteins.

The ability to select and combine desired components from a library of polyketides and postpolyketide biosynthesis genes for generation of novel polyketides is useful. The method(s) of the disclosure make it possible to, and facilitate the cloning of, novel-stable recombined polyketide synthases.

A desired stable protein developed by the methods of the disclosure can be ligated into a vector containing an expression regulatory sequences which can control and regulate the production of the protein. Use of vectors which have an exceptionally large capacity for exogenous nucleic acid introduction are particularly appropriate for use with large chimeric genes and are described by way of example herein to include the f-factor (or fertility factor) of E. coli. This f-factor of E. coli is a plasmid which affects high-frequency transfer of itself during conjugation and is ideal to achieve and stably propagate large nucleic acid fragments, such as gene clusters from mixed microbial samples.

The various techniques, methods, and aspects of the invention described herein can be implemented in part or in whole using computer-based systems and methods. Particularly, the sequence based searches, alignments, identification of crossover locations and regression analysis can be implemented by computer algorithms. In some instances the process carried out by computer may be operably connected to robotic devices for the synthesis of recombined recombinant proteins or reagents and may further include receiving stability or function data from automated assays. Additionally, computer-based systems and methods can be used to augment or enhance the functionality described above, increase the speed at which the functions can be performed, and provide additional features and aspects as a part of or in addition to those described elsewhere in this document. Various computer-based systems, methods and implementations in accordance with the above-described technology are presented below.

A processor-based system can include a main memory, preferably random access memory (RAM), and can also include a secondary memory. The secondary memory can include, for example, a hard disk drive and/or a removable storage drive, representing a floppy disk drive, a magnetic tape drive, an optical disk drive, etc. The removable storage drive reads from and/or writes to a removable storage medium. Removable storage medium refers to a floppy disk, magnetic tape, optical disk, and the like, which is read by and written to by a removable storage drive. As will be appreciated, the removable storage medium can comprise computer software and/or data.

In alternative embodiments, the secondary memory may include other similar means for allowing computer programs or other instructions to be loaded into a computer system. Such means can include, for example, a removable storage unit and an interface. Examples of such can include a program cartridge and cartridge interface (such as the found in video game devices), a movable memory chip (such as an EPROM or PROM) and associated socket, and other removable storage units and interfaces, which allow software and data to be transferred from the removable storage unit to the computer system.

The computer system can also include a communications interface. Communications interfaces allow software and data to be transferred between computer system and external devices. Examples of communications interfaces can include a modem, a network interface (such as, for example, an Ethernet card), a communications port, a PCMCIA slot and card, and the like. Software and data transferred via a communications interface are in the form of signals, which can be electronic, electromagnetic, optical or other signals capable of being received by a communications interface (e.g., information from flow sensors in a microfluidic channel or sensors associated with a substrates X-Y location on a stage). These signals are provided to communications interface via a channel capable of carrying signals and can be implemented using a wireless medium, wire or cable, fiber optics or other communications medium. Some examples of a channel can include a phone line, a cellular phone link, an RF link, a network interface, and other communications channels. In this document, the terms “computer program medium” and “computer usable medium” are used to refer generally to media such as a removable storage device, a disk capable of installation in a disk drive, and signals on a channel. These computer program products are means for providing software or program instructions to a computer system. In particular, the disclosure includes instructions on a computer readable medium for calculating the proper O.sub.2 concentrations to be delivered to a bioreactor system comprising particular dimensions and cell types.

Computer programs (also called computer control logic) are stored in main memory and/or secondary memory. Computer programs can also be received via a communications interface. Such computer programs, when executed, enable the computer system to perform the features of the disclosure including the regulation of the location, size and content substrates or products in microwells.

In an embodiment where the elements are implemented using software, the software may be stored in, or transmitted via, a computer program product and loaded into a computer system using a removable storage drive, hard drive or communications interface. The control logic (software), when executed by the processor, causes the processor to perform the functions of the invention as described herein.

In another embodiment, the elements are implemented primarily in hardware using, for example, hardware components such as PALs, application specific integrated circuits (ASICs) or other hardware components. Implementation of a hardware state machine so as to perform the functions described herein will be apparent to person skilled in the relevant art(s). In yet another embodiment, elements are implanted using a combination of both hardware and software.

The following EXAMPLES are provided to further illustrate but not limit the invention.

### EXAMPLES

The versatile cytochrome P450 family of heme-containing redox enzymes hydroxylates a wide range of substrates to generate products of significant medical and industrial importance. A particularly well-studied member of this diverse enzyme family, cytochrome P450 BM3 (CYP102A1, or “A1”) from Bacillus megaterium, has been engineered extensively for biotechnological applications that include fine chemical synthesis and producing human metabolites of drugs. In an effort to create new biocatalysts for these applications, structure-guided SCHEMA recombination of the heme domains of CYP102A1 and its homologs CYP102A2 (A2) and CYP102A3 (A3) was used to create 620 folded and 335 unfolded chimeric P450 sequences made up of eight fragments, each chosen from one of the three parents. Chimeras are written according to fragment composition: 23121321, for example, represents a protein which inherits the first fragment from parent A2, the second from A3, the third from A1, and so on. A survey of the activities of 14 chimeras demonstrated that the sequence diversity created by SCHEMA recombination also generated functional diversity, including the ability to accept substrates not accepted by any of the parents.

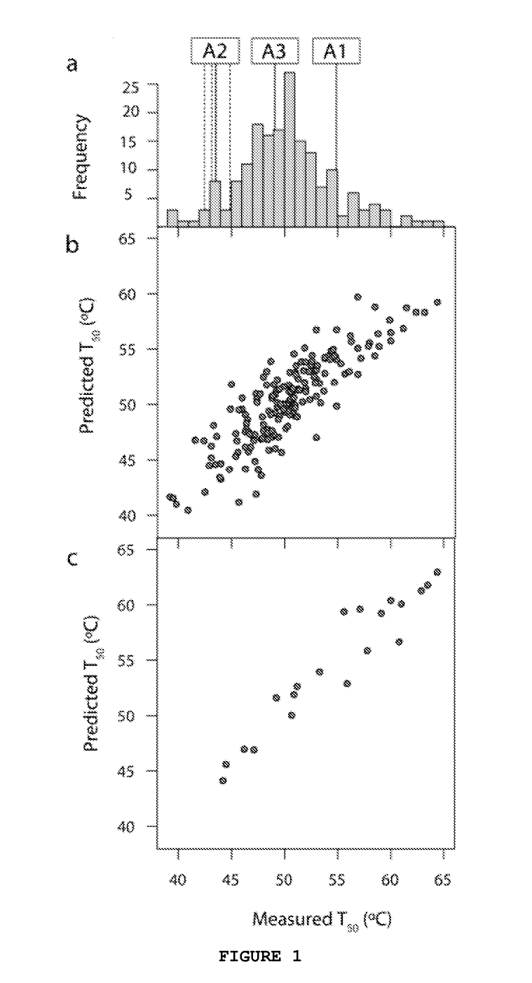

Most mutations (including those made by recombination) are destabilizing; thus most of the chimeras will be less stable than the most stable parent. Of the thousands of new P450s in the library, choosing those with the greatest stability for detailed characterization of activities and specificities is important. To do so, the thermostabilities of 184 P450 chimeras (Table 3) were measured in the form of T₅₀, the temperature at which 50% of the protein irreversibly denatured after incubation for ten minutes. Folded chimeras that were expressed at sufficient levels for the stability analysis and exhibited denaturation curves that could be fit to a two-state denaturation model were selected. The parental proteins have T₅₀ values of 54.9° C. (A1), 43.6° C. (A2) and 49.1° C. (A3) (FIG. 1a). This sample of the folded P450s contains many that are more stable than the most stable parent (A1) (FIG. 1a).

The contribution of block-additive thermostability effects were assessed by analyzing the T₅₀ values of the 184 chimeric P450s with linear regression. Regression of T₅₀ against chimera fragment composition revealed a strong linear correlation between predicted and observed T₅₀ over all 184 chimeras: Pearson r=0.856 (FIG. 1b) (Table 4).

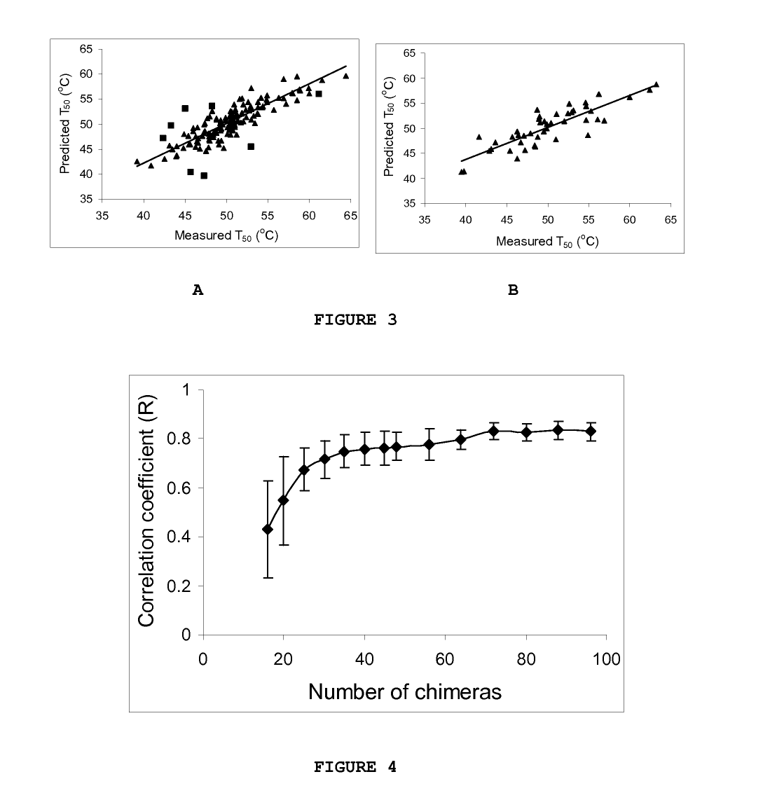

To examine whether the results allow generalization from one data subset to another and to address the possibility of over-fitting, the data was randomly divided into a training set (139 data points) and a test set (45 data points). The standard deviations of regression (σ_{R}) and measurement (σₘ=1.0° C.) were used to guide the data training. After each training cycle, every data point was weighted in terms of its role in determining the regression line. If the prediction error (the temperature difference between the predicted T₅₀ and measured one) of a data point was more than 2σ_{R}, it was removed. When a_{R} was less than 2σ_{M} (2.0° C.), the training process stopped. After two training cycles, a σ_{R} of 1.9° C. was achieved. After removing only 8 outliers, r for the training set was improved from 0.847 to 0.892 (FIG. 3a). When the trained regression parameters (Table 4) were used to predict thermostabilities of proteins in the test data set, the correlation was r=0.857, validating the regression model (FIG. 3b). The linear regression model was further confirmed by 10-fold cross-validation.

The most thermostable P450 (MTP) chimera predicted by the model parameters obtained from the training set would have a T₅₀ of 63.8° C. and fragment composition 21312333. This sequence was constructed, expressed and characterized; its T₅₀ of 64.4° C., within measurement error of the predicted value, made it 9.5° C. more stable than the most thermostable parent, A1. It was in fact the most stable of all the more than 230 chimeras that have been characterized to date. To further test the model predictions, the T₅₀ values of 19 additional chimeras from the 620 folded chimeras were measured, seven predicted to be highly thermostable and twelve picked at random (Table 3). Predicted and measured T₅₀ values for all 20 new P450s, including the MTP, correlated extremely well (r=0.956) (FIG. 1c).

In the absence of noise, one may fully determine an N-parameter regression model using only N specific measurements. In the presence of noise, additional measurements will tend to increase the accuracy of the predictions. A certain number of sequences from the 204 chimeras with measured T₅₀s were randomly selected and the ability of regression models tested based on these sequences to predict the T₅₀s of the remaining chimeras. By using a large randomized training set the effect of experimental noise was reduced. Equally important, by training on chimeras scattered throughout the sequence space biasing the resulting regression model to a single reference state was avoided. About 35 to 40 measurements were found to be sufficient for accurate predictions of chimera stability, although slight improvements in prediction accuracy could be seen with more data points (FIG. 4).

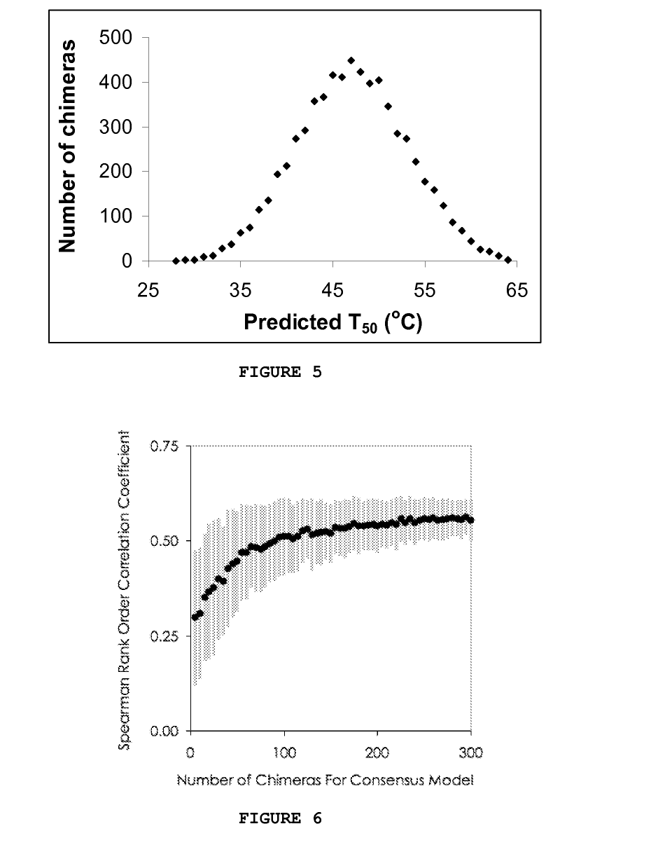

Linear regression model parameters obtained from the 204 T₅₀ measurements (Table 4) were then used to predict T₅₀ values for all 6,561 chimeras in the library (FIG. 5). A significant number (˜300) of chimeras are predicted to be more stable than A1. Those with predicted T₅₀ values greater than or equal to 60° C. (total of 30) were used for construction and further characterization. Five were already generated in our previous work⁴; the remaining 25 were constructed. As shown in Table 1, all 30 predicted stable chimeras were stable, with T₅₀ between 58.5° C. and 64.4° C. The stability predictions were quite accurate, with root mean square deviations between the predicted and measured T₅₀ values of 1.6° C., close to the measurement error (1.0° C.).

TABLE 1
Parent cytochrome P450 heme domains and 44 stabilized chimeras constructed by
recombination of stabilizing fragments.

| | Predicted | Measured | Consensus | Relative | | Predicted | Measured | Consensus | Relative |
|---|---|---|---|---|---|---|---|---|---|
| Sequence | T₅₀ (° C.) | T₅₀ (° C.) | energy | activity* | Sequence | T₅₀ (° C.) | T₅₀ (° C.) | energy | activity* |
| 11111111 | 44.8 | 54.9 | 0.000 | 1.0 | 22312313 | 60.6 | 61.0 | −2.324 | 2.5 |
| 22222222 | N/A | 43.6 | N/A | 0.5 | 21313313 | 60.6 | 64.4 | −2.324 | 4.7 |
| 33333333 | 45.1 | 49.1 | −1.013 | 0.2 | 21312133 | 60.5 | 60.1 | −2.832 | 2.8 |
| 21312333 | 63.8 | 64.4 | −3.247 | 1.0 | 22311331 | 60.4 | 58.9 | −1.603 | 5.1 |
| 21312331 | 62.8 | 60.6 | −3.057 | 3.1 | 22312231 | 60.3 | 61.4 | −2.790 | 2.3 |
| 21311333 | 62.8 | 59.2 | −1.994 | 2.5 | 21313231 | 60.3 | 61.0 | −2.791 | 1.8 |
| 21312233 | 62.7 | 63.1 | −3.181 | 0.6 | 22311233 | 60.3 | 60.9 | −1.727 | 3.1 |
| 22312333 | 62.4 | 63.5 | −3.045 | 1.9 | 21311311 | 60.0 | 61.0 | −1.083 | 3.2 |
| 21313333 | 62.4 | 62.9 | −3.046 | 3.8 | 22313331 | 60.0 | 58.5 | −2.655 | 7.2 |
| 21312313 | 62.0 | 62.2 | −2.525 | 2.8 | 21312211 | 59.9 | 59.3 | −2.270 | 2.8 |
| 21311331 | 61.8 | 62.9 | −1.805 | 1.0 | 22313233 | 59.9 | 60.4 | −2.779 | 5.7 |
| 21312231 | 61.7 | 62.8 | −2.991 | 1.0 | 21212333 | 59.6 | 63.2 | −3.120 | 0.4 |
| 21311233 | 61.7 | 62.7 | −1.928 | 0.7 | 21112333 | 59.5 | 61.6 | −3.202 | 1.1 |
| 21313331 | 61.4 | 62.2 | −2.856 | 5.5 | 22313231 | 58.9 | 59.0 | −2.589 | 6.3 |
| 22312331 | 61.4 | 59.3 | −2.856 | 5.1 | 21212233 | 58.5 | 60.0 | −3.055 | 1.3 |
| 22311333 | 61.4 | 60.1 | −1.793 | 4.7 | 21112331 | 58.5 | 61.6 | −3.013 | 0.6 |
| 22312233 | 61.3 | 61.0 | −2.980 | 2.7 | 21111333 | 58.5 | 62.4 | −1.950 | 2.6 |
| 21313233 | 61.3 | 60.0 | −2.980 | 3.3 | 21112233 | 58.4 | 58.7 | −3.137 | 0.7 |
| 21312311 | 61.0 | 59.1 | −2.336 | 3.0 | 22212333 | 58.2 | 58.2 | −2.919 | 3.2 |
| 22313333 | 61.0 | 64.3 | −2.845 | 9.0 | 22112333 | 58.1 | 58.0 | −3.001 | 4.2 |
| 21311313 | 61.0 | 61.2 | −1.273 | 2.7 | 21113333 | 58.1 | 61.0 | −3.002 | 4.1 |
| 21312213 | 60.9 | 60.6 | −2.459 | 1.1 | 23313333 | 57.1 | 61.2 | −2.433 | 2.0 |
| 21312332 | 60.8 | 59.9 | −2.739 | 1.3 | 22112233 | 57.0 | 58.7 | −2.935 | 5.2 |
| 21311231 | 60.7 | 63.2 | −1.739 | 0.8 | | | | | |

*Relative activity on 2-phenoxyethanol, reported as total turnover number normalized to that of the most active parent (A1).
N/A: Due to library construction bias, T₅₀ could not be predicted or the consensus energy calculated for heme domains containing fragment A2 at position 4.

TABLE 2
Thermostable chimeras are active on drugs not accepted by the parent enzymes.
a. Products of biotransformations on verapamil.

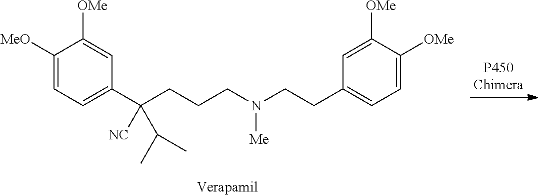

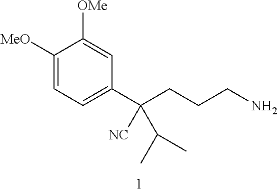

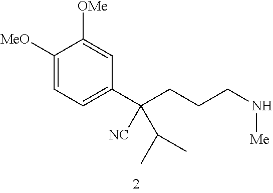

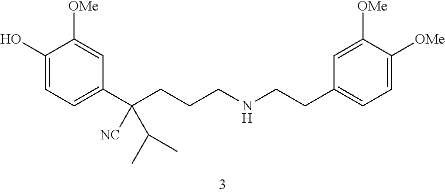

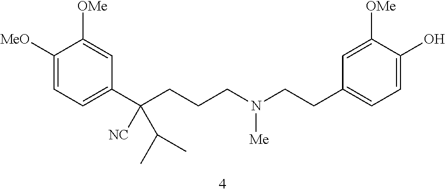

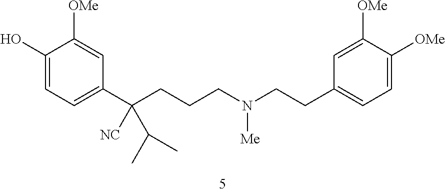

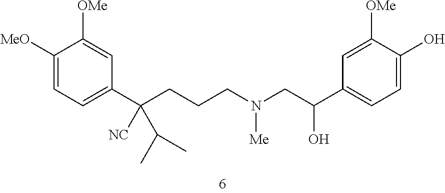

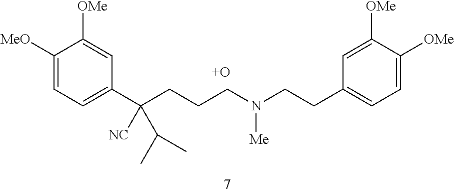

| Chimera | % Conversion* | %1 | %2 | %3 | %4 | %5 | %6 | %7 |
|---|---|---|---|---|---|---|---|---|
| 21312332 | 6 | | 33 | 17 | 17 | | | 33 |
| 21313331 | 5 | | 20 | 20 | 20 | | 20 | 20 |
| 21113333 | 5 | | 20 | | 40 | | 20 | 20 |
| 22313231 | 43 | | 32 | | | 47 | 5 | 16 |
| 22313333 | 34 | 15 | 20 | | | 41 | 9 | 15 |

b. Products of biotransformations on astemizole.

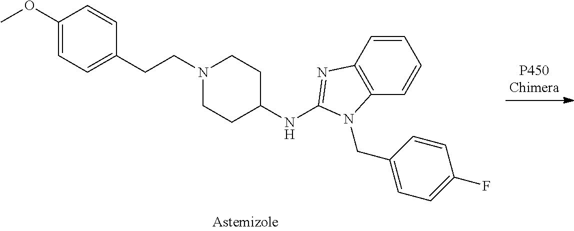

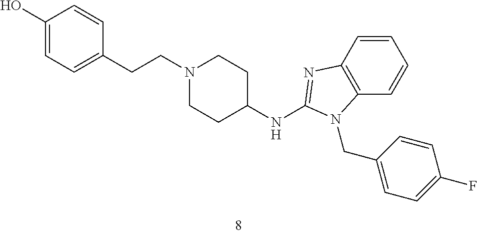

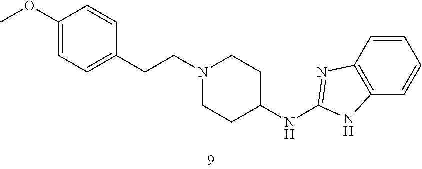

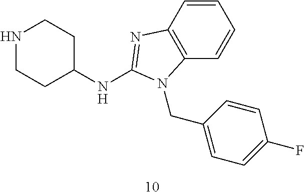

| Chimera | % Conversion* | %8 | %9 | %10 |
|---|---|---|---|---|
| 22313231 | 9 | 45 | 33 | 22 |
| 22313333 | 9 | 56 | 22 | 22 |

*200 μL reactions were run at 25° C. for 2 h using clarified lysate containing 2.5 μM P450 chimera, 250 μM drug and 1 mM hydrogen peroxide.

TABLE 3
T₅₀ values and sequences of 204 chimeric cytochromes P450. The first
184 chimeras are those for data training and testing, and the last 20 chimeras
(bold) are those used to test the linear regression model.

| Sequence | T₅₀ (° C.) | Sequence | T₅₀ (° C.) | Sequence | T₅₀ (° C.) | Sequence | T₅₀ (° C.) |
|---|---|---|---|---|---|---|---|
| 32233232 | 39.8 | 32312322 | 49.1 | 32212231 | 47.4 | 23213333 | 56.1 |
| 32313233 | 52.9 | 32312231 | 52.6 | 23212212 | 48.0 | 21333233 | 54.2 |
| 21133233 | 48.8 | 21232332 | 49.3 | 22113223 | 49.9 | 22233212 | 44.0 |
| 31312113 | 45.0 | 31331331 | 47.3 | 22233211 | 46.3 | 21313112 | 54.8 |
| 21332223 | 48.3 | 21132222 | 45.6 | 23213311 | 49.5 | 31213233 | 50.6 |
| 21312323 | 61.5 | 21212333 | 63.2 | 31212321 | 44.9 | 22132113 | 40.6 |
| 22312322 | 54.6 | 21231233 | 50.6 | 23112233 | 51.0 | 31112333 | 55.7 |
| 21212112 | 51.2 | 22212322 | 50.7 | 32332323 | 48.5 | 31212331 | 51.8 |
| 23133121 | 47.3 | 21112122 | 50.3 | 22112223 | 52.8 | 22232222 | 47.5 |
| 11312233 | 51.6 | 22111223 | 51.3 | 32313231 | 52.5 | 23332221 | 46.4 |
| 21133312 | 45.4 | 23233212 | 39.5 | 32132232 | 42.5 | 21332131 | 58.5 |
| 21133313 | 50.8 | 31312212 | 48.9 | 22232233 | 49.6 | 23231233 | 45.5 |
| 11332233 | 43.3 | 32211323 | 46.6 | 22232322 | 45.4 | 22111332 | 50.9 |
| 31212332 | 53.4 | 21213231 | 54.9 | 22333211 | 50.7 | 23312121 | 49.3 |
| 12211232 | 49.1 | 21332312 | 52.9 | 22332223 | 52.4 | 22332222 | 50.3 |
| 31312133 | 52.6 | 22332211 | 53.0 | 23213212 | 49.0 | 23312323 | 53.8 |
| 12232332 | 39.2 | 22113323 | 53.8 | 23333213 | 50.1 | 21131121 | 53.0 |
| 22133232 | 47.9 | 22113332 | 48.7 | 31312233 | 57.9 | 32212232 | 48.8 |
| 22233221 | 46.8 | 22213132 | 52.0 | 22232333 | 53.7 | 22112323 | 55.3 |
| 23113323 | 51.0 | 31213332 | 50.8 | 31333233 | 46.5 | 21232232 | 49.5 |
| 11332212 | 47.8 | 22113211 | 51.1 | 22213212 | 50.5 | 11212333 | 50.4 |
| 32332231 | 49.4 | 22313323 | 60.0 | 22132212 | 46.6 | 31212232 | 51.0 |
| 22132331 | 53.3 | 32333233 | 47.2 | 21332233 | 58.9 | 23213211 | 47.4 |
| 23313111 | 56.9 | 22331223 | 51.7 | 23333131 | 50.5 | 11331312 | 43.5 |
| 23112323 | 46.0 | 23333233 | 51.0 | 31312332 | 54.9 | 23331233 | 50.9 |
| 11113311 | 51.2 | 22333332 | 49.0 | 21333221 | 51.3 | 22133323 | 49.4 |
| 21232233 | 50.6 | 23332331 | 48.0 | 22333223 | 49.9 | 33333233 | 46.3 |
| 12332233 | 47.1 | 21233132 | 42.4 | 21111333 | 62.4 | 22233323 | 48.4 |
| 23333311 | 45.7 | 13333211 | 45.7 | 12212212 | 44.8 | 32232131 | 43.9 |
| 32132233 | 42.9 | 22232331 | 50.5 | 11313233 | 48.3 | 31312323 | 52.3 |
| 22331123 | 47.9 | 22313233 | 58.5 | 32113232 | 47.9 | 21313313 | 64.4 |
| 12212332 | 48.4 | 31311233 | 56.9 | 21113322 | 50.4 | 22333231 | 53.1 |
| 31212323 | 48.7 | 21132321 | 49.3 | 31313232 | 51.9 | 22232123 | 43.1 |
| 21132323 | 50.1 | 21132212 | 48.8 | 31332233 | 49.9 | 21312123 | 60.8 |
| 23332231 | 51.4 | 23313233 | 56.3 | 21133232 | 46.4 | 23133311 | 44.2 |
| 12112333 | 50.9 | 21332322 | 48.8 | 22112211 | 54.7 | 22113111 | 49.2 |
| 22133212 | 47.2 | 22132231 | 53.0 | 21333333 | 58.0 | 23212211 | 50.7 |
| 31113131 | 54.9 | 21113312 | 53.0 | 22213223 | 50.8 | 21212321 | 53.3 |
| 23313333 | 61.2 | 22312223 | 56.2 | 21332112 | 50.4 | 21333211 | 55.9 |
| 21113133 | 51.9 | 23332223 | 46.7 | 21331332 | 52.0 | 22232212 | 46.2 |
| 21111323 | 54.4 | 32212323 | 48.4 | 11313333 | 53.8 | 23313323 | 50.9 |
| 22212123 | 47.7 | 21212111 | 57.2 | 32311323 | 52.0 | 32312333 | 57.8 |
| 12211333 | 50.6 | 31212212 | 47.1 | 23132231 | 48.0 | 12313331 | 51.2 |
| 23113112 | 46.3 | 22232121 | 49.7 | 12232232 | 40.9 | 21311331 | 62.9 |
| 21313122 | 50.5 | 21232212 | 47.8 | 21212231 | 59.9 | 21313231 | 61.0 |
| 23112333 | 54.3 | 21333223 | 49.1 | 33312333 | 54.7 | 22312133 | 57.1 |
| 12213212 | 44.0 | 23213232 | 48.5 | 22313232 | 58.8 | 22312231 | 60.0 |
| 23132233 | 43.6 | 22113232 | 51.1 | 22312111 | 53.0 | 22312311 | 55.6 |
| 21313311 | 56.9 | 11331333 | 46.3 | 32212233 | 49.9 | 22312332 | 59.1 |
| 21332231 | 60.0 | 22333321 | 49.2 | 21132112 | 47.1 | 22312333 | 63.5 |
| 23133233 | 43.1 | 21232321 | 46.0 | 23132311 | 44.5 | 21312333 | 64.4 |

TABLE 4
Thermostability contribution from each fragment
calculated by linear regression.

| | Relative thermostability contribution (° C.) | | |
|---|---|---|---|
| Regression | 184 chimeras | 139 chimeras | 204 chimeras |
| coefficient | (no training) | (with training) | (with training) |
| a₀ | 47.0 | 46.0 | 44.8 |
| a₁₂ | 7.2 | 7.1 | 7.6 |
| a₁₃ | 1.4 | 1.2 | 1.5 |
| a₂₂ | −1.3 | −1.2 | −1.4 |
| a₂₃ | −4.5 | −5.4 | −5.3 |
| a₃₂ | −0.2 | −0.1 | 0.1 |
| a₃₃ | 3.7 | 4.1 | 4.3 |
| a₄₃ | −5.8 | −5.4 | −5.9 |
| a₅₂ | 0.2 | 1.1 | 1.0 |
| a₅₃ | −0.7 | −0.4 | −0.4 |
| a₆₂ | 1.4 | 1.4 | 2.2 |
| a₆₃ | 2.5 | 2.2 | 3.3 |
| a₇₂ | −1.4 | −2.3 | −2.5 |
| a₇₃ | 2.1 | 1.8 | 1.8 |
| a₈₂ | −2.9 | −2.0 | −2.0 |
| a₈₃ | −0.5 | 0.6 | 1.0 |

Note:
The thermostability contribution of each fragment shown is relative to the corresponding fragment from parent A1, which was used as the reference.

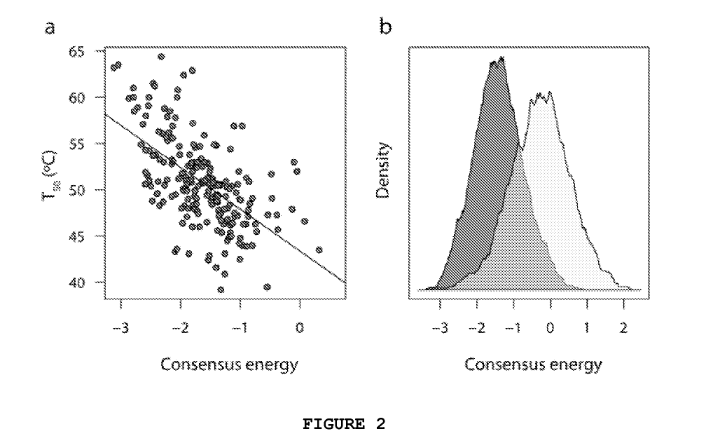

The multiple sequence alignment of the folded chimeras were then tested to determine whether they can be used predict the stable sequences, similar to ‘consensus stabilization’ methods based on natural sequence alignments. The stability of each chimera was estimated from the collection of folded chimeras. Lower consensus energies were observed to be associated with higher T₅₀ values (FIG. 2a; Pearson r=−0.58, P<<10⁻⁹). Furthermore, folded proteins tend to have lower consensus energies than unfolded ones (FIG. 2b; Wilcoxon signed rank test P<<10⁻⁹).

The tradeoff between the number of chimera sequences used to calculate the energies and the statistical error associated with ranking chimeras by consensus was examined. Random subsets containing 5, 10, 15 . . . 300 sequences from the 613 folded chimeras were selected and the consensus energies calculated for the three parents and 204 chimeras with known T₅₀s. The Spearman rank correlation coefficient (rₛ) was then calculated between the consensus energy predictions and the measured T₅₀ values. This process was repeated 10 times, and calculated the average r, and standard deviation for each sample size (FIG. 6). The average rank-order correlation coefficient is reliably above 0.5 (with standard deviations values less than 0.1) when 85 or more chimera sequences are used.

Having demonstrated that sequence and folding status alone can be used to make nontrivial predictions of relative stability, the most stable chimeras were then predicted. The consensus energy for each chimera fragment was calculated (Table 5). The total consensus energies of all 6,561 chimeras in the library were calculated; the 20 with the lowest consensus energies are listed in Table 6. A total of 17 of these top 20 (8 of which had already been constructed based on linear regression prediction) were generated. Five additional chimeras that were predicted to be stable and were constructed are also included in Table 1. All 44 chimeras that were constructed for this study are more stable than the most stable parent, have predicted T₅₀s above measured T₅₀ of the most stable parent, and are also predicted to be more stable based on consensus energy.

TABLE 5
Consensus energy contribution from each fragment.

| | Unselected | Selected | | Relative | Error |
|---|---|---|---|---|---|
| | Frequency (625 | Frequency | | Consensus | Estimate |
| Fragment | chimeras) | (644 chimeras) | Bias | Energy | (+/−) |
| x₁₁ | 64 | 53 | 0.31 | 0.00 | 0.58 |
| x₁₂ | 266 | 416 | 1.28 | −0.64 | 0.18 |
| x₁₃ | 295 | 175 | 1.42 | 0.33 | 0.26 |
| x₂₁ | 191 | 253 | 0.92 | 0.00 | 0.26 |
| x₂₂ | 218 | 236 | 1.05 | 0.20 | 0.25 |
| x₂₃ | 216 | 155 | 1.04 | 0.61 | 0.29 |
| x₃₁ | 156 | 163 | 0.75 | 0.00 | 0.32 |
| x₃₂ | 201 | 192 | 0.96 | 0.09 | 0.28 |
| x₃₃ | 268 | 289 | 1.29 | −0.03 | 0.21 |
| x₄₁ | 249 | 330 | 0.80 | 0.00 | 0.20 |
| x₄₂ | N/A | N/A | N/A | N/A | N/A |
| x₄₃ | 376 | 314 | 1.20 | 0.46 | 0.16 |
| x₅₁ | 168 | 67 | 0.81 | 0.00 | 0.44 |
| x₅₂ | 220 | 308 | 1.06 | −1.26 | 0.22 |
| x₅₃ | 237 | 269 | 1.14 | −1.05 | 0.23 |
| x₆₁ | 184 | 143 | 0.88 | 0.00 | 0.32 |
| x₆₂ | 230 | 253 | 1.10 | −0.35 | 0.24 |
| x₆₃ | 211 | 248 | 1.01 | −0.41 | 0.25 |
| x₇₁ | 208 | 169 | 1.00 | 0.00 | 0.29 |
| x₇₂ | 241 | 182 | 1.16 | 0.07 | 0.27 |
| x₇₃ | 176 | 293 | 0.84 | −0.72 | 0.26 |
| x₈₁ | 169 | 185 | 0.81 | 0.00 | 0.30 |
| x₈₂ | 272 | 217 | 1.31 | 0.32 | 0.23 |
| x₈₃ | 184 | 242 | 0.88 | −0.18 | 0.27 |

N/A = not applicable, due to bias against chimeras containing fragment x₄₂ in the SCHEMA library.

The sequence with the highest-frequency fragments at all eight positions, chimera 21312333, is called the consensus sequence. It has the lowest consensus energy and is predicted to be the most stable. In fact, 21312333 has the highest measured stability among all 238 chimeras with known T₅₀ and is also the MTP predicted by the linear regression model. The consensus sequence obtained by analyzing the alignment of multiple folded chimeras differs substantially from that obtained by simply examining the three parental sequences and designating the consensus fragment as that which differs the least from the other two parents (21221332).

The stability predictions were sufficiently accurate to identify both sequencing errors and point mutations in the chimeras. The sequences of P450 chimeras were originally determined by DNA probe hybridization, which has a ˜3% error rate; small numbers of point mutations during library construction are also expected. The 13 chimeras were re-sequenced with prediction error of more than 4° C. from the original set of 189 chimeras whose T₅₀s were measured and analyzed by linear regression. Five either had incorrect sequences or contained point mutations (Table 7); they were eliminated from the subsequent analyses.

TABLE 6
The 20 chimeras with lowest total consensus
energies.

| Sequence | Consensus energy | Sequence | Consensus energy |
|---|---|---|---|
| 21312333 | −3.247 | 21113333 | −3.002 |
| 21112333 | −3.202 | 22112333 | −3.001 |
| 21312233 | −3.181 | 21312231 | −2.991 |
| 21112233 | −3.137 | 21313233 | −2.980 |
| 21212333 | −3.120 | 22312233 | −2.980 |
| 21312331 | −3.057 | 21112231 | −2.947 |
| 21212233 | −3.055 | 21113233 | −2.936 |
| 21313333 | −3.046 | 22112233 | −2.935 |
| 22312333 | −3.045 | 21212331 | −2.931 |
| 21112331 | −3.013 | 22212333 | −2.919 |

TABLE 7
Sequence errors and mutations identified by linear
regression.

| | | | | Predicted | Predicted |
|---|---|---|---|---|---|
| | | | | T₅₀ (° C.) | T₅₀ (° C.) |
| Original | Correct | | Measured | (wrong | (correct |
| sequence | sequence | Mutation | T₅₀ (° C.) | sequence) | sequence) |
| 31312333 | 33332333 | no | 47.4 | 57.9 | 46.5 |
| 32333232 | 22333232 | no | 53.5 | 44.6 | 51.6 |
| 22131221 | 22131223 | no | 51.0 | 44.7 | 45.8 |
| 22212321 | same | P40L | 47.9 | 53.7 | — |
| 22312232 | same | Q354P | 53.4 | 58.1 | — |

Note:
T₅₀s were not predicted for chimeras containing point mutations.

Further work also showed that both the regression and consensus models perform well enough to significantly increase the odds of identifying sequencing errors and mutations. The chimeras 22313333, 21311311, and 22311333 were predicted to be highly stable while they had been reported unfolded⁴. Full sequencing showed that the original 22313333 construct was incomplete and missing some fragments; the original 21311311 construct had an insertion; 22311333 had two point mutations leading to two amino acid substitutions. After correction, all three chimeras are very stable (Table 1).

The newly constructed thermostable chimeras and corrected sequences were added to the previously published sequence-folding status data (Table 8). The consensus analysis using the corrected sequence-folding data (of 644 folded chimeras) versus 238 chimeras with measured T₅₀s was re-performed. The correlation r between consensus energy and measured thermostability improved significantly, from −0.58 to −0.67.

TABLE 8
Additional folded chimeric cytochrome P450 heme
domain sequences generated by the methods of the disclosure.
21311231
21311233
21312133
21312231
21312233
21312311
21312332
21313233
21313331
21313333
22311233
22312233
22313231
22312331
21312331
21312313
21312333
22311333
21112333
21112233
21113333
21112331
22112333
22112233
21312213
22311331
21212233
22212333
21311313
22313333
21311311
22311333

An enzyme's half-life of (irreversible) inactivation (t_{1/2}) is commonly used to describe stability. The t_{1/2} at 57° C. for 13 stable chimeras and the three parents were measured (Table 9). The results show that the increased stability can have a profound effect on half-life: while the most stable parent, A1, lost its ability to bind CO with a half-life of 15 min at this temperature, chimera 21312231 had a half-life of 1600 min, or more than 108 times greater. The MTP and the consensus chimera 21312333 similarly has a very long half-life of 1550 min. T₅₀ has also been shown to correlate linearly with urea concentrations required for half-maximal denaturation for variants of CYP102A1. Therefore, The stable P450 chimeras can also be more tolerant to inactivation by chemical denaturants.

TABLE 9
Half-lives of inactivation (t_{1/2}) at 57° C. of three
parent proteins and 13 stabilized chimeric proteins.

| Sequence | t_{1/2} (min) | Sequence | t_{1/2} (min) | Sequence | t_{1/2} (min) | Sequence | t_{1/2} (min) |
|---|---|---|---|---|---|---|---|
| 11111111 | 15 | 22312331 | 170 | 22312233 | 400 | 21312233 | 980 |
| 22222222 | 0.36 | 21313233 | 160 | 22312231 | 140 | 22312333 | 670 |
| 33333333 | 0.86 | 21312331 | 110 | 21313331 | 390 | 22313333 | 150 |
| 21313333 | 350 | 21313231 | 930 | 21312231 | 1600 | 21312333 | 1550 |

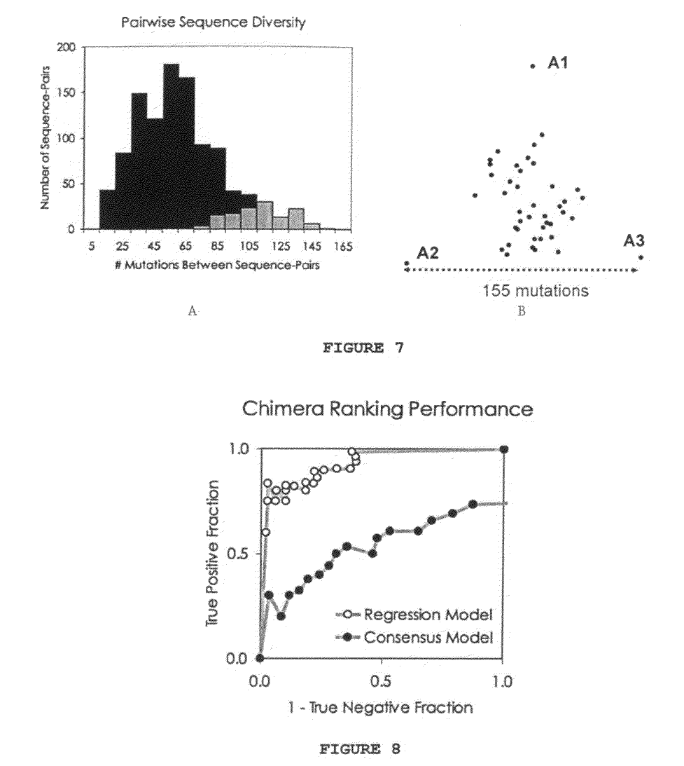

All 44 stable chimeras were verified by full sequencing to eliminate any possibility that the enhanced thermostabilities were due to mutations, insertions or deletions. The stable chimeras comprise a diverse family of sequences, differing from one another at 7 to 99 amino acid positions (46 on average) (FIG. 7). The distance to the closest parent is as high as 99 amino acids. The expression levels of most of the thermostable chimeras were higher than those of the parent proteins. Most thermostable chimeras expressed well even without the inducing agent isopropyl-beta-D-thiogalactopyranoside (IPTG).

To determine whether the stable chimeras retained catalytic activity and, more importantly, whether they acquired new activities of biotechnological importance, The peroxygenase activity measurements of the thermostable chimeras on 2-phenoxyethanol, a substrate on which all three parent enzymes are active, showed that all 44 chimeras are active (Table 1). Furthermore, many of them were more active than the most active parent (A1). The thermostable chimeras were also tested for activity on two drugs, verapamil and astemizole, and measured the extent of metabolite formation by HPLC/MS with higher order MS analysis. While none of the parents showed activity on either drug, three chimeras produced significant quantities of metabolites for verapamil, and two chimeras produced metabolites from both verapamil and astemizole. Products 2, 4, 5, 8 and 10 (Table 2) are known human metabolites and are the products of reactions with the human CYP3A4, 1A2, 2C and 2D6 enzymes.

The disclosure and data demonstrate two approaches to predicting protein stability using different data. One is performed by linear regression of sequence-stability data, and the other is based on consensus analysis of the multiple sequence alignment. The best prediction approach depends on the target protein and the relative ease with which folding status and stability are measured. The linear regression model uses stability data, which are often more difficult to obtain than a simple determination of folding status. The linear regression model, however, also requires fewer measurements and always predicted more true positives with fewer false positives than the consensus approach based on folding status (FIG. 8).

Consensus stabilization is based on the idea that the frequencies of sequence elements correlate with their corresponding stability contributions. This correlation is typically assumed to follow a Boltzmann-like exponential relationship¹⁵. Such a relationship is most sensible if, in analogy to statistical mechanics, the sequences are randomly sampled from the ensemble of all possible folded P450s. Natural sequences are related by divergent evolution and may not comprise such a sample. Our chimeric protein data set, in contrast, represents a large and nearly random sample of all the 6,561 possible chimeras. Support for the fundamental assumptions underlying consensus stabilization approaches: sequence elements contribute additively to stability, stabilizing fragments occur at higher frequencies among folded sequences, and the consensus sequence is the most stable in the ensemble are provided by the data. These results demonstrate the tolerance of the consensus stabilization idea to different ensembles (chimeric libraries versus evolved families) and sequence changes (recombination versus stepwise mutation). Unlike previous implementations of consensus stabilization, however, the approach described here generates dozens of stable proteins, and these proteins differ from each other and from the parents at many amino acid residues.

The high degree of additivity observed may appear surprising, considering the cooperative nature of protein folding and the many tertiary contacts in the native structure. The additivity of stability changes to proteins has long been known. Non-additive effects are expected when sequence changes are coupled or result in significant structural changes. Structural disruption is less likely in chimeras than with random mutants because all sequence elements are believed to fold to a similar structure in at least one context, that of the parental sequence. Furthermore, such block-additivity may be maximized by the library design, which reduces coupling. SCHEMA identifies sequence fragments that minimize the number of contacts, or interactions, that can be broken upon recombination. Two residues in a chimera are defined to have a contact if any heavy atoms are within 4.5 Å; the contact is broken if they do not appear together in any parent at the same positions. Among a total of about 500 contacts for a P450 chimera, an average of fewer than 30 were broken for the sequences in the SCHEMA library. The SCHEMA fragments that were swapped in this library have many intra-fragment contacts; the inter-fragment contacts are either few or are conserved among the parents. As a result, the fragments function as pseudo-independent structural modules that make roughly additive contributions to stability. The additivity was strong enough to enable detection of sequencing errors based on deviations from additivity, prediction of thermostabilities for uncharacterized chimeras with high accuracy, and prediction of the T₅₀ of the most stable chimera to within measurement error. Because SCHEMA effectively identifies functional chimeras with other protein scaffolds, such as β-lactamases²², this approach should allow one to identify novel stable, functional sequences for other protein families.

Both approaches demonstrated here identify highly stable sequences; recombination ensures that they also retain biological function and exhibit high sequence diversity by conserving important functional residues while exchanging tolerant ones. This sequence diversity can give rise to useful functional diversity. Assembly of the stable P450 chimeras was motivated in part by a desire to generate new or improved P450 activities in a stable catalyst framework. This study demonstrated improvements in activity (on 2-phenoxyethanol) as well as acquisition of entirely new activities (on verapamil and astemizole) in the stabilized enzymes. That the P450 chimeras can produce authentic human metabolites of drugs opens the door to rapid drug metabolic profiling and lead diversification using soluble enzymes that are produced efficiently in E. coli.

The disclosure demonstrates that chimeric proteins exhibit a broad range of stabilities, and that stability of a given folded sequence can be predicted based on data (either stability or folding status) from a limited sampling of the chimeric library. By assembling predicted stable sequences, 44 stabilized P450s were generated that differ significantly from their parent proteins, are expressed at high levels, and are catalytically active. Individual members of the stable P450 family exhibit activity on biotechnologically relevant substrates. This approach allows the creation of whole families of stabilized proteins that retain existing functions and also explore new functions.

Thermostability measurements. Cell extracts were prepared and P450 concentrations were determined as reported previously⁴. Cell extract samples containing 4 μM of P450 were heated in a thermocycler over a range of temperatures (from 36° C. to 75° C.) for 10 minutes followed by rapid cooling to 4° C. for 1 minute. The precipitate was removed by centrifugation. The P450 remaining in the supernatant was measured by CO-difference spectroscopy. T₅₀, the temperature at which 50 percent of protein irreversibly denatured after a 10-min incubation, was determined by fitting the data to a two-state denaturation model⁸. To check the variability and reproducibility of the measurement, four parallel independent experiments (from cell culture to T₅₀ measurement) were conducted on A2, which yielded an average T₅₀ of 43.6° C. and a standard deviation (σ_{M}) of 1.0° C. For some sequences, T₅₀s were measured twice, and the average of all the measurements was used in the analysis.

Linear regression. The linear model

$${T}_{50} = {a}_{0} + {\sum }_{i} {\sum }_{j} {a}_{ij} {x}_{ij}$$

was used for regression, where T₅₀ is the dependent variable and fragments xᵢⱼ (from the i^{th} position and j^{th} parent, where i=1, and j=2 or 3) are the independent variables. The were dummy-coded, such that if a chimera has fragment 1 from parent 2, x₁₂=1 and x₁₃=0. Parent A1 was used as the reference for all eight positions, so the constant term (a₀) is the predicted T₅₀ of A1 and the regression coefficients aᵢⱼ represent the thermostability contributions of fragments xᵢⱼ relative to the corresponding reference (A1) fragments. In general, the reference fragment at each of the 8 positions can be chosen arbitrarily. Due to construction bias, the fragment from parent A2 at position 4 is almost completely missing from the data set. the few chimeras having this fragment were therefore deleted from all analyses, including consensus analysis. Regression was performed using SPSS(SPSS for Windows, Rel. 11.0.1. 2001. Chicago: SPSS Inc.).

Consensus energy calculation. Assuming the frequency of a fragment at position i is exponentially related to its stability contribution and that these fragment contributions are additive, total chimera consensus energy relative to a reference sequence can be calculated from

$${Δɛ}_{total} ∝ {\sum }_{i} - ln \frac{{f}_{i}}{{f}_{i , ref}} ,$$

where f_{i,ref} is the ensemble frequency of the fragment at i in a reference sequence. A1 was again used as the reference, so that A1 has consensus energy of zero; the choice of reference sequence is arbitrary and does not influence the results. Note that the values reported are actually proportional to energy differences from the reference; referred to as consensus energies for brevity. The raw frequencies fᵢⱼ^{raw} of fragment i from parent j in the folded ensemble may reflect biases in the assembly of chimeras from their constituent fragments. Bias can be assessed by measuring the frequencies fᵢⱼ^{unselected in an unselected set of sequences to determine the biases b}ᵢⱼ=nₚₐᵣₑₙₜₛfᵢⱼ^{unselected}, which in an unbiased ensemble will be equal to 1. For the P450 ensemble the fᵢⱼ^{unselected} are known (Table 5). Construction bias can be corrected directly by dividing the fᵢⱼ^{raw} by the bᵢⱼ, and bias-corrected frequencies were used in all analyses.

Construction of thermostable chimeric cytochrome P450s. To construct a given stable chimera, two chimeras having parts of the targeted gene (e.g. 21311212 and 11312333 for the target chimera 21312333) were selected as templates. The target gene was constructed by overlap extension PCR, cloned into the pCWori expression vector, and transformed into the catalase-free E. coli strain SN0037. All constructs were confirmed by fully sequencing.

Enzyme activity assays. Activity on 2-phenoxyethanol was measured as reported previously with slight modifications. 80 μl of cell lysate containing 4 P450 chimera was mixed with 20 μl of 2-phenoxyethanol solution (60 mM) in each well of a 96-well plate. The reaction was initiated by adding 20 μl of hydrogen peroxide (120 mM). Final concentrations were: 2-phenoxyethanol, 10 mM; hydrogen peroxide, 20 mM. After 1.5 h, the reactions were quenched with 120 μL urea (8M in 200 mM NaOH) before adding 36 μL 4-aminoantipyrine (0.6%). Mixtures were blanked on the plate reader at 500 nm before adding 36 μL potassium peroxodisulfate (0.6%). After 10 min of color development, the solutions were re-measured for absorbance. Absorbances were normalized to the most active parent A1.

Biotransformations with verapamil and astemizole. 60 μL of cell lysate containing ˜8.3 μM P450 chimera was mixed with 90 μL of EPPS buffer (0.1M, pH 8.2) and 10 μL drug (5 mM). The reaction was initiated by addition of 40 μL hydrogen peroxide (5 mM). Final concentrations were: drug, 250 μM; hydrogen peroxide, 1 mM. After 1.5 h, the reaction was quenched with 200 μL acetonitrile and the mixtures centrifuged 10 min at 18000 g. 25 μL supernatant was analyzed by HPLC. Conditions with solvent A (0.2% formic acid (v/v) in H₂O) and solvent B (acetonitrile) used to elute the products of metabolism at 200 uL/min were: 0-3 min, A:B 90:10; 3-25 min, linear gradient to A:B 30:70; 25-30 min, linear gradient to A:B 10:90. Samples whose chromatograms contained more than the parent drug peak were further analyzed by LCMS and MS/MS. Identical conditions to the HPLC method detailed above were used for the LC portion of the analysis followed by MS operation in positive ESI mode. MS/MS spectra were acquired in a data dependent manner for the most intense ions. Product identification was accomplished by comparison of retention times and tandem MS spectra against controls from rat liver microsomes. HPLC separations were performed using a Supelco Discovery C18 column (2.1×150 mm, 5μ) on a Waters 2690 Separation module in conjunction with a Waters 996 PDA detector. LCMS and MS/MS spectra were obtained using the ThermoFinnigan LCQ classic at the Caltech MS facility.

A number of embodiments have been described. Nevertheless, it will be understood that various modifications may be made without departing from the spirit and scope of the description. Accordingly, other embodiments are within the scope of the following claims.

1. A method for generating one or more stabilized proteins, comprising:
identifying a plurality of parental polypeptides (P) which are evolutionary, structurally or evolutionary and structurally related, such that the Parental polypeptides have a degree of similarity or identity of at least 60%;
selecting a set of crossover locations comprising a number (N) of peptide segments in at least a first parental polypeptide and at least a second parental polypeptide of the plurality of parental polypeptides;
generating a sample set of less than (P^{N}) recombinant proteins comprising peptide segments from each of the at least first parental polypeptide and the at least second parental polypeptide;
measuring the stability of the sample set of to identify expressed and stably folded recombinant proteins;
performing regression analysis and/or consensus analysis on the expressed and stably folded, recombinant proteins in order to identify stability-associated peptide segments;
generating a stabilized polypeptide comprising the stability-associated peptide segments; and
measuring the activity and/or stability of the stabilized polypeptide.

2. The method of claim 1, wherein the stabilized polypeptide comprises an enzyme.

3. The method of claim 2, wherein the enzyme is selected from the group consisting of carbohydrases, alpha-amylase, β-amylase, cellulase, β-glucanase, β-glucosidase, dextranase, dextrinase, glucoamylase, hemmicellulase/pentosanase/xylanase, invertase, lactase, pectinase, pullulanase, proteases, oxygenases, acid proteinase, alkaline protease, pepsin, peptidases, aminopeptidase, endo-peptidase, subtilisin, lipases and esterases, aminoacylase, glutaminase, lysozyme, penicillin acylase, isomerase, oxireductases, alcohol dehydrogenase, amino acid oxidase, catalase, chloroperoxidase, peroxidase, lyases, acetolactate decarboxylase, aspartic β-decarboxylase, histidase, transferases, and cyclodextrin glycosyltransferase.

4. The method of claim 1, wherein the stabilized polypeptide is a therapeutic protein.

5. The method of claim 1, wherein the selecting a set of crossover locations comprises:
aligning the sequences of the plurality of parental polypeptides; and
identifying regions of sequence identity.

6. The method of claim 5, wherein the method comprises sequence alignment and structural relatedness data obtained from one or more methods selected from the group consisting of X-ray crystallography, NMR, searching a protein structure database, homology modeling, de novo protein folding, and computational protein structure prediction.

7. The method of claim 1, wherein the selecting a set of crossover locations comprises:
identifying a number of coupling interactions between of residues in the at least first parental polypeptide with residues in the at least second parental polypeptide;
generating a plurality of data structures, wherein each data structure represents a crossover chimera comprising a recombination of the at least first parental polypeptide and the at least second parental polypeptide, and wherein each data structure has a recombination at a different location;
determining, for each data structure, a crossover disruption value, which correlates to the number of coupling interactions disrupted in the crossover chimera of the data structure; and
identifying, among the plurality of data structures, a particular data structure having a crossover disruption value which is below a certain cutoff value, wherein the crossover location of the crossover chimera as identified by the particular data structure is a crossover location.

8. The method of claim 7, wherein the coupling interactions are identified by a determination of conformational energies between residues of the at least first parental polypeptide with residues of the at least second parental polypeptide, or by a determination of interatomic distances between residues of the at least first parental polypeptide with residues of the at least second parental polypeptide.

9. The method of claim 8, wherein the conformational energies are determined from a three-dimensional structure of the at least first parental polypeptide and of the at least second parental polypeptide.

10. The method of claim 8, wherein the interatomic distances are determined from a three-dimensional structure of the at least first parental polypeptide and of the at least second parental polypeptide.

11. The method of claim 7, wherein the coupling interactions between residues are identified by having an absolute value of interaction energy between the residues above a defined threshold value.

12. The method of claim 7, wherein the cutoff value is calculated from the average level of crossover disruptions for the plurality of data structures.

13. The method of claim 5, wherein the identifying regions of sequence identity further comprises identifying possible cut points in the polypeptides based upon the regions of sequence identity.

14. The method of claim 5, wherein the regions of sequence identity must contain at least 4 residues.

15. The method of claim 1, wherein P^{N} is greater than 50.

16. The method of claim 1, wherein measuring of stability comprises a technique selected from the group consisting of chemical stability measurements, functional stability measurements and thermal stability measurements.

17. The method of claim 1, wherein the regression analysis comprises analyzing sequence-stability data and wherein the consensus analysis comprises analyzing multiple sequence alignment (MSA) of folded versus unfolded proteins.

18. The method of claim 17, wherein the sequence-stability data comprises sequence information operably associated with stability measurements.

19. The method of claim 17, wherein the analyzing sequence-stability data can be performed using the following equation:
$${T}_{50} = {a}_{0} + {\sum }_{i} {\sum }_{j} {a}_{ij} {x}_{ij} ,$$
where T₅₀ is the dependent variable and peptide segments xᵢⱼ (from the i^{th} position and from the j^{th} parental polypeptide are the independent variables), wherein the constant term (a₀) is the predicted T₅₀ of a parental polypeptide and the regression coefficients aᵢⱼ represent the thermostability contributions of peptide segment xᵢⱼ relative to the corresponding reference peptide segment of the parental polypeptide.

20. The method of claim 17, wherein the consensus analysis comprises sequence information of stabilized polypeptides and a frequency of stability-associated peptide segments.

21. The method of claim 20, wherein the consensus analysis comprises measuring the frequency of a stability-associated peptide segment at a position (i) in a stabilized protein and exponentially valuing the position:segment repeats to give a consensus energy value.

22. The method of claim 21, wherein stability-associated peptide segments that promote stability reduce the overall consensus energy value of a stabilized protein can be expressed as
$${Δɛ}_{total} ∝ {\sum }_{i} - ln \frac{{f}_{i}}{{f}_{i , ref}} ,$$
wherein the overall consensus energy value (Δεₜₒₜₐₗ) can be determined by assuming the frequency (f) of a fragment at position (i) as it relates to the ensemble frequency of the fragment at position (i) in a reference sequence (f_{i,ref}) is exponentially related to its stability contribution and that these fragment contributions are additive.

23. The method of claim 1, wherein the analysis comprises a combination of sequence-stability data and consensus analysis of multiple sequence alignment (MSA) of folded versus unfolded proteins.

24. A method for generating one or more stabilized proteins, comprising:
selecting crossover locations in a sample set of a plurality of parental polynucleotides (P) encoding polypeptides that are evolutionary, structurally or evolutionary and structurally related, such that the polypeptides have a degree of similarity or identity of at least 60%, wherein the set of crossover locations defines a number (N) of oligonucleotide segments each segment encoding a peptide;
performing recombination between a subset, less than P^{N}, of the parental polynucleotides having crossover locations to obtain a sample set of recombinant proteins comprising peptide segments encoded by the oligonucleotide segments;
measuring the stability of the sample set for expressed and stably folded recombinant proteins;
performing regression analysis and/or consensus analysis on the expressed stably folded recombinant proteins in order to identify stability-associated peptide segments and the encoding oligonucleotide segment;
generating a stabilized polypeptide encoded by a combination of oligonucleotide encoding stability-associated peptide segments; and
measuring the activity and/or stability of the stabilized polypeptide.

25. A method of identifying stability-associated peptide fragments, comprising:
selecting crossover locations in a sample set of a plurality of parental polynucleotides (P) encoding polypeptides that are evolutionary, structurally or evolutionary and structurally related, such that the polypeptides have a degree of similarity or identity between the polypeptides of at least 60%, wherein the set of crossover locations defines a number (N) of oligonucleotide segments each segment encoding a peptide;
performing recombination between a subset, less than P^{N}, of the parental polynucleotides having crossover locations to obtain a sample set of recombinant proteins comprising peptide segments encoded by the oligonucleotide segments;
measuring the stability of the sample set of to identify expressed and stably folded recombinant proteins;
performing regression analysis and/or consensus analysis on the expressed and stably folded recombinant proteins in order to identify stability-associated peptide segments and the encoding oligonucleotide segment;
outputting sequence data and stability measurements for stability-associated peptide segments to a database, wherein the database comprises both nucleotide and amino acid sequences.

26. A database of stability-associated peptide segments with stability values obtained from the method of claim 59 comprising a query and output to user function.

27. The method of claim 1 that is automated.

28. The method of claim 1, wherein the determining of crossover locations and/or regression analysis is determined by a computer.

29. A computer implemented method comprising:
selecting crossover locations in a sample set of a plurality of parental polynucleotides (P) encoding polypeptides that are evolutionary, structurally or evolutionary and structurally related, such that the polypeptides have a degree of similarity or identity of at least 60%, wherein the set of crossover locations defines a number (N) of oligonculeotide segments each segment encoding a peptide;
performing recombination between a subset, less than P^{N}, of the parental polynucleotides having crossover locations to obtain a sample set of recombinant proteins comprising peptide segments encoded by the oligonucleotide segments;
obtaining stability measurement data from the sample set to identify expressed and stably folded recombinant proteins;
performing regression analysis and/or consensus analysis on the expressed and stably folded recombinant proteins in order to identify stability-associated peptide segments and the encoding oligonucleotide segment;
generating a stabilized polypeptide encoded by a combination of oligonucleotide encoding stability-associated peptide segments; and
outputting the sequence of the stabilized polypeptide to a user interface.
